(19) United States
(12) Patent Application Publication
(10) Pub. No.: US 2018/0369064 A1
(43) Pub. Date: Dec. 27, 2018

(54) DEVICES AND METHODS FOR INCREASED BLOOD FLOW, HEALING, AND PAIN CONTROL

(71) Applicant: MMJ LABS, LLC, Atlanta, GA (US)

(72) Inventors: Amy L. BAXTER, Atlanta, GA (US); Loius A. CALDERON, Atlanta, GA (US)

(21) Appl. No.: 16/115,484

(22) Filed: Aug. 28, 2018

Related U.S. Application Data
(63) Continuation-in-part of application No. 15/148,258, filed on May 6, 2016, now Pat. No. 10,058,476.
Application No. 15/148,258 is a continuation of application No. 13/795,683, filed on Mar. 12, 2013, now Pat. No. 9,333,144.
Application No. 13/795,683 is a continuation-in-part of application No. 13/426,330, filed on Mar. 21, 2012, now Pat. No. 8,740,960.
Application No. 13/426,330 is a continuation of application No. 11/538,718, filed on Oct. 4, 2006, now Pat. No. 8,147,533.

(51) Int. Cl. A61H 23/00; A61F 7/10; A61M 5/42; A61F 7/02; A61F 7/03; A61F 7/00

(52) U.S. Cl. CPC A61H 23/00; A61F 7/10; A61M 5/422; A61H 2201/0207; A61F 2007/0285; A61F 2007/0084; A61F 2007/0078; A61F 2007/0075; A61F 2007/0001; A61H 2201/0214; A61H 2209/00; A61F 7/032

(57) ABSTRACT

A device comprising a casing; at least one vibratory source; at least one power source; at least one switch to actuate the vibratory source; electrical communication between the vibratory source, the power source, and the switch; and an optional thermal element for modulating the temperature of a contacted area of a surface. Also disclosed herein are methods for using the disclosed device.

### CROSS REFERENCE TO RELATED APPLICATIONS

This application is a continuation-in-part of U.S. patent application Ser. No. 15/148,258 filed May 6, 2016, which is a continuation of U.S. patent application Ser. No. 13/795,683 filed Mar. 12, 2013, and issued as U.S. Pat. No. 9,333,144 on May 10, 2016, which is a continuation-in-part of U.S. patent application Ser. No. 13/426,330 filed Mar. 21, 2012, and issued as U.S. Pat. No. 8,740,960 on Jun. 3, 2014, which is a continuation of U.S. patent application Ser. No. 11/538,718 filed Oct. 4, 2006, and issued as U.S. Pat. No. 8,147,533 on Apr. 3, 2012, each of which is herein incorporated in their entireties.

### TECHNICAL FIELD

The present invention generally relates to devices and methods for the improvement, of circulation, for example in blocked ducts, veins or arteries, healing of injured sites in a subject, for example, musculoskeletal injuries or chronic wounds, and for pain control and irritating sensation control, such as itching and burning, by interfering with transmission of nerve signals by, for example, aDelta nerves, using devices capable of vibration with or without thermal effects.

### BACKGROUND

Vibration has long been used to decrease the pain of dental injections, but vibrating needles have not been found to be effective in other dermal situations. Use of a simple hand-held vibrating massager has decreased injection pain in adults, and with the adjuvant of cold therapy was tested in both children and adults and found to relieve pain (Baxter et al. 2011; Baxter et al. 2009; Inal and Kelleci 2012).

Increasing vein diameter (vasodilation) is extremely important for venipuncture in children, as well as for circulation in adults. Venipuncture success in emergency department is regularly found to be between 55 and 65% (Taddio et al, 2005; Spandorfer et al. 2005) and can be as low as 49% in children (Hess 2010).

Moreover, injured sites in a subject comprise acute or chronic conditions in which normal cellular arrangements or metabolism are disturbed. Such injured sites are detrimental to the subject, for example in creating pain or disability, and are also a major healthcare cost. For example, lower back pain and injury healing represent significant healthcare problems. Chronic wounds are a source of cost and disability, especially in diabetic subjects. Approximately 25% of the 150 million diabetics worldwide will experience a chronic wound, and the majority of these wounds will remain unhealed after 20 weeks of treatment. Normal wound healing comprises overlapping periods of inflammation, angiogenesis, tissue remodeling and tissue formation. Inflammation can decrease and delay healing and increase pain. For injuries, common treatment is ice and elevation to reduce inflammation, but cold alone can diminish angiogenesis and remodeling. For skin healing and ulceration, treatments such as negative pressure bandages or ultrasound have been used to treat impaired tissue regeneration, but the efficacy of such treatments to stimulate angiogenesis and remodeling is debated.

Devices in the prior art that have sought to induce vasodilation have used dramatically different means, including electrical stimulation and subsequent muscle contraction (US 2011/0071595), vacuum suction (U.S. Pat. No. 5,454,778), and compression (U.S. Pat. No. 6,129,688). Moreover, none of these devices are intended for increasing healing, the success of venipuncture or reducing pain and discomfort associated with low blood flow.

Therefore, there is a need for an intentional agent (device and/or method) to increase vasodilation contributing to the success of venipuncture, improve blood flow, promote healing, and decrease claudication (pain from inadequate blood flow). Such an intentional device may include vibration and/or stimulation, along with thermal actions, and optionally, have a shape suitable to conform to the body. Such a device may be reusable, easy to clean, tolerable to children and adults, and may be optionally configured for use with or without external power sources, such as in the developing world (for example, operation with a power cord, with a battery, with a solar or light cell, or without the need for external power). As ease of use is advantageous in this environment, embodiments that include rechargeable options, moveable locations, optional numbers of vibration units, or incorporation of thermal elements, cold and/or heat.

Accordingly, there is a need for a device and method that allows for the local improvement of blood flow, improvement of circulation in blocked ducts, veins or arteries, improved healing, and for pain control and irritating sensation control. There also is a need for a device and method using vibration or a combination of vibration and a thermal element that is applied to a site in a subject to treat pain, burning or itching sensations and promote healing. These needs and others are met by the various aspects of the present invention.

### SUMMARY

In various aspects, the present invention relates to methods and devices for increasing local blood flow, promoting healing, and pain control and irritating sensation control. In an exemplary aspect, the methods and devices provide increasing local blood flow and flow through ducts, veins or arteries, promoting healing and for pain control and irritating sensation control, such as itching and burning, by interfering with transmission of nerve signals by, for example, aDelta nerves, using devices capable of vibration and/or thermal effects. A method of the present invention comprises contacting a device of the present invention near or at a site of restricted blood flow, for example, to small veins or a site of vasospasm, initiating vibration and/or thermal effects, for a time sufficient to effect an increase in the diameter of the blood vessels, such as veins or arteries, and reducing the blockage in ducts. In another exemplary aspect, the methods and devices provide for treating injured sites in a subject, such as for injury and wound healing and tissue repair, using devices capable of vibration and/or thermal effects. In another exemplary aspect, a method disclosed herein comprises contacting a device disclosed herein near, adjacent to or directly to an injured site of a subject, and initiating vibration and/or thermal effects, for a time sufficient to effect an increase in healing of the injured site.

In another exemplary aspect, a method of the present invention comprises reducing the pain or irritating sensation, such as those caused by an injected medication, comprising contacting a device of the present invention between the spinal cord and the site where the pain or irritating sensation is initiated, such as a site of injection of a medication that causes a burning or painful sensation when injected; initiating vibration by the device in an intermittent or continuous vibration, optionally applying cold simultaneously with the vibration, vibrating for a sufficient time to interfere with nerve transmission as the injection is occurring, moving the device to the site of injection once the medication in injected, and initiating vibration at the injection site for a time sufficient to reduce the pain felt from the injection site optionally applying cold simultaneously to the injection site, to interfere with transmission of pain signals by aDelta nerves. The sensations from an injected medication, such as bunting or itching, are different from those produced by needle stick pain, and the burning and/or itching sensations are not found with all medications, whereas needle stick pain is generally found with all injections or needle sticks.

In another exemplary aspect, a method of the present invention comprises reducing an itching sensation in an animal, comprising contacting a device of the present invention to a site of itching, initiating vibration by the device, optionally applying a thermal effect simultaneously with the vibration, and reducing the sensation of itching, for example, by interfering with the transmission of nerve signals by aDelta nerves at the site of itching.

In another exemplary aspect, a method of the present invention comprises treating blocked mammary ducts, comprising, contacting a device of the present invention at or adjacent to a site of a blocked mammary duct, initiating vibration by the device, optionally providing a thermal effect simultaneously with the vibration, and modulating the condition of the blocked duct, for example, by at least releasing a portion of the blockage and/or relieving the pain caused by the blockage.

In another exemplary aspect, a method disclosed herein comprises treating one or more injured sites in a subject, such as for treating an injured site or area by providing wound healing and/or tissue repair, comprising contacting a device disclosed herein on or adjacent to an injured site of a subject, such as a chronic wound, a burn, an acute or overuse injury, myofascial injury or inflammation; initiating vibration by the device in an intermittent or continuous vibration (which provides stimulation to the application site), optionally applying cold or heat simultaneously with the vibration, vibrating for a predetermined time, and optionally moving the device to another injured site or to a second location adjacent to the first injured site. For example, for an acute or overuse injury, a device may be applied directly to the injury site. For example, at sites wherein skin integrity is disrupted, a method may comprise placing a device adjacent to the disrupted site. At sites where the skin integrity is not disrupted, a method may comprise placing a device directly on the site.

In another exemplary aspect, a method disclosed herein comprises accelerating healing of one or more injured sites in a subject, such as for wound healing and tissue repair, comprising contacting a device disclosed herein on or adjacent to an injured site of a subject, such as an acute or chronic injury or chronic wound or a burn; initiating vibration by the device in an intermittent or continuous vibration, optionally applying cold or heat simultaneously with the vibration, vibrating for a predetermined time, and optionally moving the device to another injured site or to another location adjacent to the first injured site.

In further aspects, various methods disclosed herein may be effective for both reducing pain and enhancing wound healing or repair at injured sites. By way of non-limiting example, by enhancing wound healing, there is generally a reduction in pain at that site. For example, by treating myofascial injury, myofascial pain is reduced. Methods of enhancing wound or tissue healing also comprise methods for reducing pain at an injured site. Methods of the present disclosure comprise methods for reducing pain associated with wounds or injured sites by applying a device disclosed herein near, adjacent to or on a wound or injured site, initiating thermal and/or vibratory effects to relieve pain associated with the wound or injured site.

According to various aspects of the present disclosure, a device of the present invention comprises a casing, which may be shaped to conform to the contour of a surface. Such a shaped casing may have an established shape, for example, by a preformed casing, or may be a moldable casing that is shaped by a user. A casing may contain a vibratory element, and optionally a thermal element may be associated with the casing. The disclosed devices comprise a casing that contain a vibratory element, and optionally a thermal element. In an aspect, a casing or at least one surface of a casing, is shaped to fit a curved surface of the body. For example, according to further aspects, one surface of a casing may be concave, shaped like the inner surface of a circle, and when the device contacts a surface, such as an arm, the concave surface of the casing substantially contacts the arm surface, meaning that a majority of the concave surface is in contact with the area of the surface. This contact of substantially the entire concave surface of the device allows for enhanced transfer of vibration and/or thermal effect to the surface. In still further aspects, one or more surfaces of a casing may be convex, shaped like the outer surface of a circle, and when the device contacts a surface, such as the back, the convex surface of the casing substantially contacts the surface, meaning that a majority of the convex surface is in contact with the area of the surface. In yet further aspects, this contact of substantially the entire convex surface of the device allows for enhanced transfer of vibration and/or thermal effect to the surface.

Vibration effects can be provided by any of the known vibratory devices such as, for illustrative purposes, a vibratory motor or an eccentric flywheel motor provided within the casing. Once vibration is initiated by providing power to the vibratory source, such as a vibratory motor, the vibration may be constant and continual, or the vibration may be intermittent, and cycle on and off at the same or a different vibration speed or frequency. Though not wishing to be bound by any particular theory, it is believed that intermittent vibration may aid in reducing or preventing habituation by the body to the vibrations. In further aspects, constant vibration during a treatment period may provide benefits to healing. One of skill in the art is able to determine which treatment method, constant or intermittent vibration, is suitable for a particular subject or ailment to be treated.

An exemplary embodiment of the device comprises a casing or moldable shape casing housing the various components of the invention and an optional strap or thermal pouch (e.g. neoprene) for holding the device to the subject. The casing may be manufactured of a stiff material to transmit vibration, and may be placed into a more flexible or pliant material in the form of a covering. The casing can be any shape, and preferably conforms to most body parts, particularly fingers, arms, shoulders, lower back, hip, feet, and legs. For example, an application area may be concave or convex so to conform to rounded areas of the body to which the device may be applied. Any other shape may be employed, so long as the shape is large enough and structured so as to be able to contain the various working components of the invention. A device of the present invention may optionally comprise an adhesive area on a portion or substantially all of the proximal side of an application area or a thermal element for affixing the proximal side of the casing/thermal element to a surface, such as the body or skin of a subject.

A method of the present invention comprises providing a device of the present invention externally to the skin surface of a subject. For example, the subject may be a human or animal to whom a hypodermic needle is being applied to either remove fluid from the subject or to inject a composition into the subject. The vibratory device of the present invention may be placed at a site of injection, injury, pain, itching, bunting, blocked vessels, or may be placed proximal to such sites. In some methods, the vibratory device is placed at one site for a period of time and then moved to a second site. Methods of the present invention allow for increased blood flow, reduction of pain, reduction of irritating or unpleasant sensations, such as itching or burning, increased healing, or for blood drawing or injection procedures.

These features, and other features and advantages of the present invention will be apparent to those of ordinary skill in the relevant art when the following detailed description of the preferred embodiments is read in conjunction with the appended drawings in which like reference numerals represent like components throughout the several views. The figures and the detailed description which follow more particularly exemplify these and other embodiments of the invention.

### BRIEF DESCRIPTION OF THE FIGURES

The accompanying Figures, which are incorporated in and constitute a part of this specification, illustrate several aspects and together with the description serve to explain the principles of the invention.

FIGS. 1A-1B are perspective views of an exemplary embodiment in accordance with the invention. In FIG. 1A, the device (10) is being applied to the arm (100) of a subject at a site 104 of initiation (104a), which may be a site of pain, or irritating sensation, such as burning or itching. The positioning of the device (10) on the subject is between the initiation site (104a) and a nerve plexi (not shown, but the location of which is known to those of ordinary skill in the pain alleviation field) such as between the initiation site (104a) and the proximal joint in this case the elbow (E). Thus, for the illustrated initiation site (104a) on a subject's arm (100), the device (10) is placed closer to the elbow (E) than to the wrist (W) as this location puts the device (10) in between the initiation site (104a) and the subject's brain. For example, the device 10 may be placed approximately 2.5 cm to 15 cm front initiation site (104a). In FIG. 1B, the device (10) is being applied to the arm (100) of a subject near a site 104 of injury (104b), which may be a site of pain, or irritating sensation, such as burning or itching. The positioning of the device (10) on the subject can be adjacent to or near the injury site (104b). Thus, for the injury site (104b) area on a subject's arm (100), the device (10) is shown on a portion of the site but may be placed approximately 0.5 cm to 25 cm from the injury site (104b) depending on the treatment indication and objectives.

FIG. 2 is a top view of an exemplary embodiment in accordance with the invention with both vibration and thermal (cold or hot) capabilities. A minimal embodiment of the external features of the invention comprises the casing (12) and a switch (16). Optional strap (14) can be used to hold the device on to the subject. Strap (14) can be attached to the casing (12) in any conventional manner or it can be an extension of casing (12) itself. The ends of strap (14) may have some type of connecting device (18), such as a hook and loop fastener, a clasp, a clip, snaps, magnets, adhesive, or the like for attaching the device about the subject's body part. Alternatively, if the ends of strap (14) are flexible, the ends can be tied together around the subject's body part. The casing (12) of a device may be curved in a manner to enhance proximity and, thus contact, of the device by a strap or wrap.

FIG. 3 is a bottom view of an exemplary embodiment in accordance with the invention with both vibration and thermal capabilities. The casing (12) has a peripheral bottom rim (20) that defines an application area generally designated (22). All or a portion of application area (22) may contact the subject or may contact or otherwise be in communication with a thermal element thereinbetween the application area (22) and the subject. The application area (22) comprises at least one of: thermal area (24) and vibration area (26). Although thermal area (24) and vibration area (26) area shown as discrete areas, this is for illustrative purposes only, as there need not be any physical delineation between thermal area (24), vibration area (26), and application area (22) and these areas can overlap and be coexistent as the same area, which may be referred to as application area (22). Thermal element (28) cooperates with thermal area (24) to apply cold or heat to the subject, and vibrational source (32) (not shown) cooperates with vibration area (26) to apply vibration to the subject. Thermal area (24) and vibrational area (26) can occupy the same area, and form application area (22). Thermal element (28) may be located within thermal element pocket (34).

FIG. 4 is sectional side view of an exemplary embodiment in accordance with the invention as shown along the latitudinal line 4-4′ of FIG. 2. The casing (12) may be a generally hollow structure sized to contain a thermal element (28) and a vibrational source (32). Thermal element (28) can be placed within thermal element pocket through mouth or opening (36) and can be held within thermal element pocket (34) by friction, adhesives, fasteners, or by a zipper or other type of closure on mouth or opening (36). In various aspects, the bottom wall (38) of thermal element pocket (34) may be sufficiently thin or have sufficient thermal transfer characteristics so as to allow the efficient transfer of cold or heat from thermal element (28) to the subject. The vibrational source (32) further comprises a power source (40) and wiring (42) electrically connecting vibrational source (32) and power source (40) to switch (16).

FIG. 5A is a circuit diagram of an exemplary embodiment in accordance with the invention. A representative circuit diagram for the vibrational source (32) is shown. The vibrational source (32), power source (40), and switch (16) are electrically connected in series by wiring (42). FIG. 5B is a circuit diagram of an exemplary embodiment in accordance with the present invention comprising a vibrational source (32), a push button switch (16), a battery power source (40), a control board (45), and optionally a speaker (46) and a light (47), in electrical connection in series by wiring (42).

FIGS. 6A-6B are perspective views of an exemplary embodiment in accordance with the invention having an ornamental cover (60) or a decorated casing (12). The device is depicted in the same use as FIGS. 1A-1B with the addition of an ornamental cover (60) or a decorated casing (12), for example, to distract the subject, such as a child.

FIG. 7 is a drawing of an exemplary device of the present invention showing the application area (22) of a device (10) comprising the proximal side (e.g., the side closest to the subject when in use) (30) of the casing (12) and the proximal side (29) of a (transparent) thermal element (28) coextensive with the application area wherein thermal element (28) associated with the proximal side of casing (12) by use of an attachment element (50), which is an elastic band in this figure.

FIGS. 8A-8B are drawings of another exemplary vibratory device in accordance with the present invention. In FIG. 8A, the device is placed between an initiation site (104a) of pain, burning, itching, or injury and the spinal cord. In FIG. 8B, the device is placed on the area directly adjacent to an injury site (104b).

FIG. 9A-9E show drawings of an exemplary device in accordance with the present invention having a concave shaped casing. FIG. 9A shows the front or anterior end (31) of the device (10) and its power switch (16). FIG. 9B shows the posterior or rear end (33) of the device (10) with its site indicator (52), and FIG. 9C shows the back or proximal side (30) of the device, that is contacts, or is placed proximally or adjacent to, the surface having a clip (50) for holding a thermal element (not shown) in place; FIG. 9D shows a front or distal side (37) of the device (10), and FIG. 9E shows a side view (35a or 35b) of the vibratory device (10) where the attachment element (50) comprises a clip, slightly protrudes from the posterior (lower) (33) proximal end and the on/off switch element (16) is shown at the anterior (upper) end (31).

FIG. 10 shows a method for using the disclosed devices in accordance with another exemplary embodiment of the present invention.

FIG. 11 shows a diagram of a system including a computing device for enabling operation of the disclosed devices in accordance with another exemplary embodiment of the present invention.

FIGS. 12A-12C depict views of exemplary embodiments of vibratory devices with both vibration and thermal (cold or hot) capabilities in accordance with the invention.

FIG. 13 depicts an exemplary embodiment of a vibratory device in accordance with the invention where thermal element (28) can be placed within thermal element pocket (34) through mouth or opening (36) and can be held within thermal element pocket (34) by friction, adhesives, fasteners, or by a zipper or other type of closure (91) on mouth or opening (36). Thermal element (28) and vibrational source (32) (not shown) cooperate with application area (22) to apply both vibration and thermal effects to the subject.

FIG. 14 depicts an exploded view of an exemplary embodiment of a vibratory device in accordance with the invention.

### DETAILED DESCRIPTION

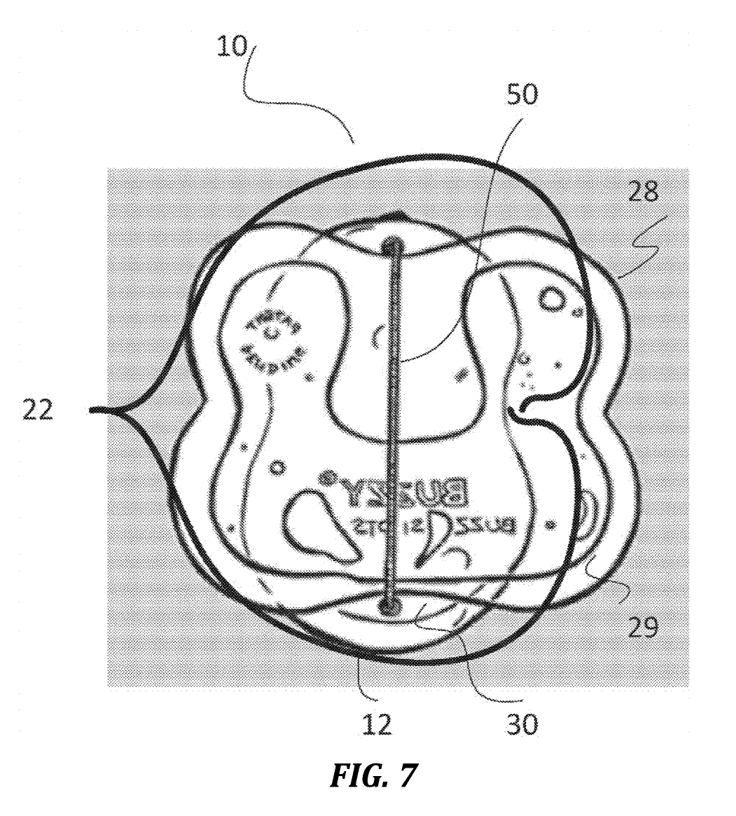

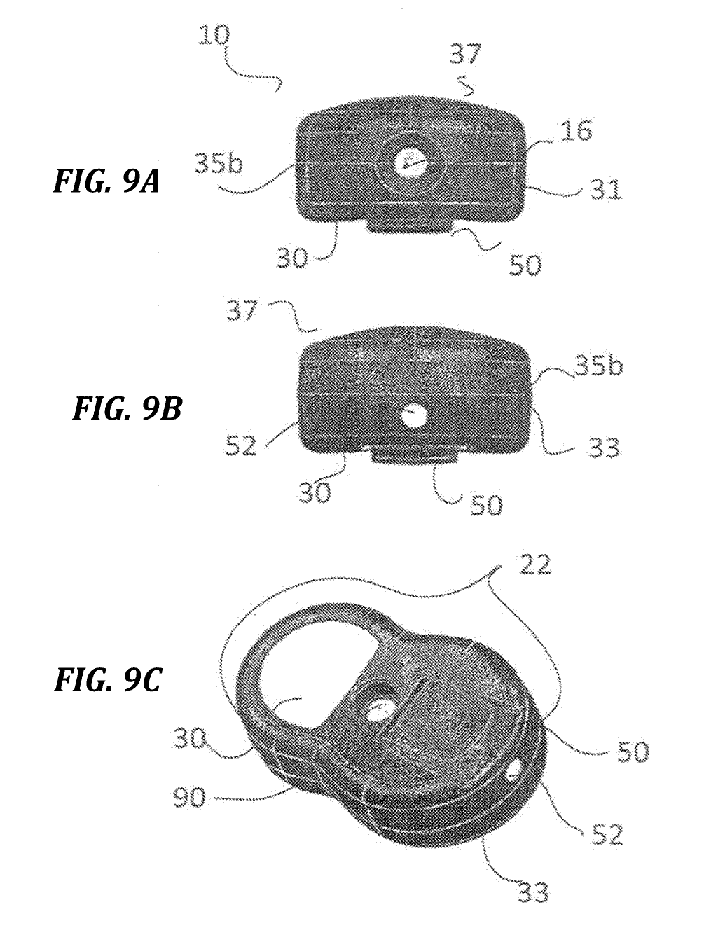

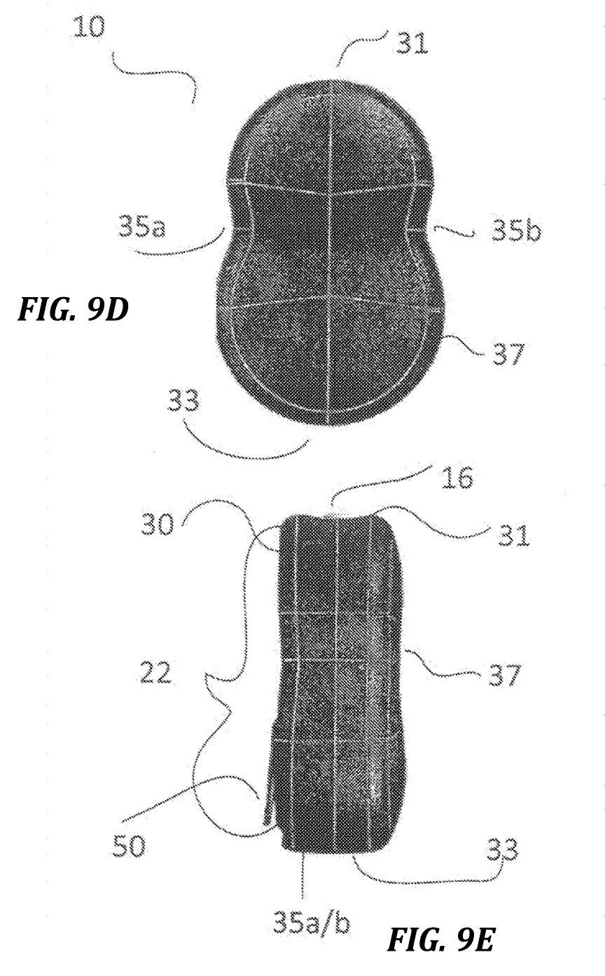

According to various aspects of the invention, disclosed herein are devices and methods for treating one or more sites in a subject, such as for reducing pain or sensation, and for increasing healing and blood or fluid flow in local areas. An embodiment of a device of the invention comprises a casing that contains a vibrational source, and an on/off switch for the vibrational source. A device may further comprise an attachment element for holding a thermal element in association with the casing. An attachment element (50) may be an integral portion of the casing, such as the clip shown in FIG. 9, or may be itself attached to the casing, such as the attachment element (50) shown in FIG. 7. An optional strap or wrap can be used to hold the device on to the subject, for example a strap that extends around a limb of the subject to secure the device on the limb of the subject such that the medical practitioner need not have an assistant present to hold the device, so that the medical practitioner can have both hands free to treat the subject, or a larger wrap that secures the vibratory device to a limb or body structure, for example, for an extended time. Further, a strap can act as a tourniquet, if necessary. Alternatively, the device can be held against the subject by the practitioner, the practitioner's assistant, or the subject.

In various aspects, a casing of a device of the present invention comprises one or more application areas. In further aspects, an application area can comprise a thermal area and/or a vibrational area. In some aspects, the application area may be the portion of the casing for contacting the surface or for contacting a thermal element that in turn contacts the surface. For simplicity of understanding, the surface may be the skin of a subject. In an aspect, the application area may be all or a portion of the proximal side of a vibratory device. A thermal element cooperates with the thermal area to apply cold and/or heat to the subject, and a vibrational source cooperates with the vibrational area to apply vibration to the subject. As used herein, a thermal element can comprise a heat element and/or cold element, and is intended to provide heat and/or cold, and one of skill can determine whether the method includes application of heat and/or cold based on the treatment objectives.

In further aspects, the position or placement of the thermal element may be variable so long as the effects of the thermal element can be felt on the subject so as to produce the desired thermal effects, such as vasodilation or vasoconstriction. In still further aspects, the position or placement of the vibrational source in the casing is variable so long as the vibrational effects of the vibrational source can be felt on the subject so as to produce the desired vibrational effects to the subject, such as, for example, to produce at least one of: vasodilation or is effective in stimulating nerves so that a pain or sensation message is blocked or interfered with in reaching the spinal cord nerves, and interfering with the perception of the pain or sensation by the subject. In yet further aspects, the walls of the casing define an interior space that, is sized to contain at least, the vibrational source, and a power source, such as batteries, and optionally a control element, a thermal element, a sound element, or a light element, and wiring to connect at least the vibrational source and the power source.

In further aspects, an attachment element, for example, a chemical fastener or a clip, panel, knob or hook, on the proximal side (30) of the device, facing the subject surface, may be used to secure a thermal element to the device. In some aspects, the thermal element may be secured to an interior surface of the proximal side (30) of the device. In still further aspects, an attachment element, such as an elastic band or thermally conductive band or panel may also be used to secure the thermal element to an interior or exterior surface of the proximal side of a device of the present invention. Adhesive on the proximal side of the casing may be used to hold a thermal element to a device. A casing may further contain a control element for controlling the operation or speed of vibration or period of vibration, for storing and providing sound, for providing a timing element, for controlling a light, such as turning a light, on or off, with or without, a timer, or making the light blink at a particular time point. In further aspects, the device may be constructed to transmit vibration and thermal effects through the attachment element, such as, for example, in embodiments where the attachment element is configured as the primary portion of the casing in contact with the patient. In still further aspects, the vibration from the vibrational source may have transmission path beginning at the vibrational source and traveling through the vibration area, the thermal element, and the attachment element before reaching the administration site of the patient. Thus, in various embodiments, the device would be able to concurrently deliver vibration and thermal effects through attachment element.

Devices and decorative casings, and the use of detractive media are disclosed in U.S. Pat. No. 8,147,533 filed Oct. 4, 2006, and issued Apr. 3, 2012, U.S. patent application Ser. No. 11/867,630, filed Oct. 4, 2007; Ser. No. 12/371,206, filed Feb. 13, 2009; and Ser. No. 13/426,330, filed Mar. 21, 2012, Ser. No. 13/795,683, filed Mar. 12, 2013; Ser. No. 14/635,540, filed Jan. 1, 2014; each of which is herein incorporated in its entirety.

In further aspects, a casing may further comprise on opening through a wall of the casing for providing an amplifier on the outer surface of the casing that is connected to a control element or a sound element contained within the casing. A casing may further comprise on opening through the casing for providing a light, such as an LED light, on the outer surface of the casing that is connected to a control element or a timing element contained within the casing. A light (and/or sound) may be turned on when vibration is initiated and turned off when power to the vibration element is turned off Alternatively, powering on the vibration element may also power on a timing element, and optionally a light (and/or sound), so that when a desired time period has occurred, the timing element may turn off the light (and/or sound), or may turn off a light (and/or sound) and the vibration element, or the timing or control element may turn on sound or light after a period of vibration. Alternatively, the timing element may be under a control that is separate from a control for the vibration element. Various device components, such as switches, control elements, such as a polycarbonate circuit board and the programming to accomplish the disclosed activities and others, and elements such as timing elements, sound elements and lights, are known, and can be selected or commercially acquired by those of skill in the art. Wires for connecting the elements within the casing or on the surface are contemplated by the present invention.

In further aspects, a casing may be shaped to provide an application area that is in contact with a surface so that substantially all of the application area contacts the surface. For example, a casing may be flat or concave in shape on at least its proximal side so that the application area is shaped so that substantially all of the proximal side of the casing contacts the surface of the surface. When a thermal element is placed on the proximal surface of the casing, substantially all of the proximal surface of the casing contacts the thermal element interposed between the casing outer surface and the contacted surface so that an area of the surface that is equivalent to the area of substantially all of the proximal side of the casing is contacted by the thermal element and receives vibrational effects therethrough (the application area). All or a portion of a casing may be curved. For example, the entire casing may be curved, such as in a concave direction (curved like the interior of a circle), so that the proximal side of the casing is contacting a surface through all or a portion of its surface (or the thermal element interposed therebetween) and the distal side (37) of the casing is curved to mirror the curve of the proximal side, so as to be comfortably held by a hand or held in place by a strap. Alternatively, only one surface, either the distal or proximal side may be curved, for example, where the proximal side is flat, but the distal side is curved. Additionally, a proximal side and/or a distal side of the casing may be flat or planar, and the lateral sides (35a or 35b) of the casing may be shaped, for example, as shown in FIGS. 7 and 9, there may be an indented area in the lateral sides. The lateral sides of the casing may be shaped in any form desired.

In further aspects, a casing may further comprise an adhesive area on a portion or substantially all of the application area. In an aspect, an adhesive area may be found on the proximal side of a thermal element and may cover a portion or substantially all of an area on the proximal side of the thermal element that corresponds to the application area of a device. A device of the present invention may optionally comprise an adhesive area on a portion or substantially all of the proximal side of the application area of the casing or the thermal element for attaching the proximal side of the device/thermal element to a surface, such as the body or skin of a subject. On the casing and/or thermal element, and before use, the adhesive area may be covered by a removable shield. In use, the removable shield is removed from the adhesive area on the proximal side of the casing and/or thermal element so that the adhesive is exposed and is capable of affixing the proximal side of a casing to a thermal element or to a surface, such as skin, or is capable of affixing the proximal side of a thermal element to a surface.

In further aspects, a surface of the casing or thermal element other than the proximal side of the device may have an adhesive or fastener area on a portion or substantially all of the surface of the device other than the proximal surface. In still further aspects, the adhesive or fastener area on such a surface may be covered by a removable shield. In some aspects, a removable shield can be removed from an adhesive area of the casing and/or thermal element so that an adhesive is exposed and is capable of affixing the side of a casing to a thermal element or to a surface, or is capable of affixing the side of a thermal element to a surface. In other aspects, a removable shield can be removed from a fastener area of the casing and/or thermal element so that a mechanical fastener is exposed and is capable of affixing the side of a casing to a thermal element or to a surface, or is capable of affixing the side of a thermal element to a surface. In yet further aspects, the disclosed devices may have one or more adhesive or fastener areas on a casing and/or on a thermal element. By way of non-limiting example, a disclosed device may have one or more adhesive or fastener areas on a casing and one or more adhesive or fastener areas on a thermal element. In some aspects, the disclosed devices may have a plurality of adhesive or fastener areas on a casing. In other aspects, the disclosed devices may have a plurality of adhesive or fastener areas on a thermal element. In still other aspects, the disclosed devices may have a plurality of adhesive or fastener areas on a casing and a plurality of adhesive or fastener areas on a thermal element. In even further aspects, alternative arrangements of adhesive or fastener areas on one or more components of the disclosed devices are contemplated by the present invention.

In further aspects, a vibrational source can be any conventional vibrational source or means for producing high frequency low amplitude vibrations. In still further aspects, a controlling or on/off switch can be a common switch or a push button on/off switch, and can be used to control operation of the vibrational source, such as to turn the vibrational source on and off. In some aspects, as further described herein, the vibrational source and other device elements may be remotely activated, such as, for example by a remote switch or wireless device, or activated by a component other than a mechanical on/off switch, such as, for example, by the removal of an impeding material, such that when the impeding material is removed, a circuit is completed and the vibrational source is activated. In other aspects, a power supply may be activated by removal of a tab such that removal of the tab allows for the completion of a circuit without the need for an on-off switch.

In further aspects, an activated vibrational source may vibrate in a constant and continuous mode, or the vibrations may be noncontinuous, such as intermittent periods or cycles of vibration and no vibration. The power source for operating the vibrational source can be any type of power source such as but not limited to a connection to an alternating current source (a wall plug), a solar or other light cell, a miniature reactor, a mechanical source such as a flywheel or springs, a disposable or rechargeable battery or the like.

In various aspects, also disclosed herein are methods of using the disclosed devices. In further aspects, a method of using a disclosed device can improve blood flow and healing at least in the area contacted by the device, through the use of vibrational and/or thermal modulation, such as of blood and/or lymph vessels, or ducts of the body. In still further aspects, in response to contacting the skin surface with a vibrating and/or thermal device of the present invention, blood flow in areas not specifically contacted by the device, such as blood flow in distal areas, may be improved, for example, in response to the increased blood flow at or near the site of contact. More specifically, the devices disclosed herein can be used for concurrently applying a combination of vibrational effect and thermal effect to a surface area of a subject. In some aspects, the combined effects of vibration and thermal heat effects can cause dilation of blood and/or lymph vessels proximal to the site of application of a device. In other aspects, the combined effects of vibration and thermal cold effects can cause constriction of blood and/or lymph vessels proximal to the site of application of a device. In yet other aspects, for ducts of the body, for example, blocked mammary ducts, the combined effects of vibration and thermal heat effects can cause a reduction in the transmission of pain signals from the blocked ducts, and also aid in reducing the blockage of the duct proximal to the site of application of a device.

By way of non-limiting example, the effects of vibration and/or thermal application may provide a local physiological effect to a surface and its underlying ducts or vessels within from 0.0 cm to 15 cm from the site of application of the device. Application of the device may have a more systemic effect by triggering a response in the local area, (0.0 cm to 15 cm) by a body part, such as a nerve, that has effects at a distant location, such as triggering a nerve response at the site of application that blocks nerve transmission of pain or sensation by spinal or other nerves. Local stimulation of blood, lymph or milk (or a body fluid in a duct) flow by a device of the present invention may alter blood, lymph, milk or duct fluid flow responses in vessels not in the area related to the surface contacted by a device of the present invention. It is known that vibration helps to reduce pain as the vibrational, or motion, nerves surmount the pain nerves, which is known as gate theory to those of ordinary skill in the field. Similarly, it is known that cold helps to reduce pain as the temperature nerves surmount the pain nerves. It also is known that warm thermal contact is effective at vasodilation. Stimulation of aDelta nerves, which is accomplished by a device of the present invention, may aid pain reduction, which may relax tenses muscles.

Though not wishing to be bound by any particular theory, it is believed that vibrational effects may act to cause release of endogenous nitric oxide which increases lumen diameter. Use of a vibratory device of the present invention aids in the removal of blocked ducts, such as mammary ducts in breastfeeding females, blocked passages such those seen in pulmonary disease, for example, in subjects with cystic fibrosis, or arteries, veins and capillaries in tissues with blocked blood flow. A method of the present invention comprises treating a subject with cystic fibrosis to open blocked pulmonary ducts or passages, comprising contacting at least one site on the outer surface of the body overlying the lungs, bronchi or trachea with a device of the present invention, initiating vibration by device for a desired time period, optionally providing thermal effects from the presence of a thermal element interposed between the device application area and the contacted surface of the subject, optionally moving the device and providing vibratory and/or thermal effects to a second area of the subject, and affecting at least one pulmonary passage or duct of a subject. For example, in small human infants having blocked pulmonary passages such an infant with cystic fibrosis, a device of the present invention may have a fiat proximal side for contacting at least a portion of the thoracic area of the infant, whether anteriorly or posteriorly or laterally on the subject, so that vibratory and/or thermal effects are provided to reduce blocked pulmonary passages and aid in unblocking clogged or blocked pulmonary passages. The gentle and directed vibrational and/or thermal effects for a controlled time period are beneficial for such blocked passages.

In further aspects, presently disclosed methods may comprise using a device disclosed herein for increasing local blood flow. In still further aspects, the present methods may comprise using a disclosed device for promoting healing. In yet further aspects, the present methods may comprise use of a disclosed device for increasing local lymph flow. In even further aspects, the present methods may comprise use of a disclosed device for increasing local duct fluid flow. A method of the present invention comprises contacting a device of the present invention to a site of restricted fluid flow, for example, to a site of small arteries or veins, vessel spasm or vasospasm, or a site of blocked or restricted lymph or duct flow, initiating vibration and/or thermal effects, for a time sufficient to effect an increase in the diameter of the vessels, such as blood vessels, such as veins and/or arteries, lymph vessels, and ducts. For example, a device of the present invention is provided to an area of the breast where the duct is blocked. For example, the device, comprising the warm thermal element, is placed within bra cup containing the breast and is held in place next to the blocked duct area by the bra. The switch is activated so as to turn on the vibration element and the heat and vibrational effects are transmitted to the blocked duct area. This treatment causes reduction in pain from the blocked ducts, and reduces the duct blockage. The treatment may continue for several minutes at a time, wherein vibration and warmth are provided by the device to the blocked duct area, and such treatments may be repeated one or more times a day for one or more days. With repeated treatment, the extent of the blocked duct is reduced and the blockage may be resolved. With reduction in blockage, pain is also reduced, but the device also functions to block pain nerve transmissions during treatment.

A method of the present invention comprises reducing the pain or burning sensation caused by injected medication. Many medications, when injected, cause a bunting or painful sensation at the site of injection. This painful injection is disturbing to the subject receiving the injection and to the medical staff who provides the medication, and reduction of these side effects of injection would be beneficial for increased compliance by the subject and reduced stress for the medical staff. A method of reducing the pain or bunting sensation of an injected medication comprises a) contacting a device of the present invention with an area on the surface of a subject between the spinal cord and the site of injection of a medication, that causes a burning or painful sensation when injected, so that at least a portion of the application area of the device contacts the area; b) initiating vibration by the device in an intermittent or continuous vibration, and optionally applying a thermal effect simultaneously with the vibration; c) as the injection continues, so as to inject the medication solution, providing vibrating and/or thermal effect for a sufficient time to interfere with nerve transmission as the injection is occurring; d) once the medication solution is injected, moving the device to the site of injection; and e) continuing or initiating vibration and/or thermal effect at the injection site for a time sufficient to reduce the pain felt from the injection site. The thermal effect may cold or warm. The device may interfere with transmission of pain signals by aDelta nerves at the site of injection. For example, a subject is injected with a medication solution of Lovenox® (enaxaprin sodium) for deep vein thrombosis treatment or in conjunction with in vitro fertilization treatment, in a lateral abdominal area. This medication is known to cause pain or burning at injection. Immediately prior to injecting the needle into a pinched area of skin (subcutaneously), a device of the present invention comprising a cold thermal element is contacted with a first contact site which is the surface of the subject's skin anteriorly/laterally to the injection site, and within a 5-25 cm range from the site of injection. The device is switched on and continuous vibration and cold is provided to the contacted area. The Lovenox® solution is injected completely into the site. The device of the present invention is moved from its first contact site to the injection site and continuous vibration and cold is provided to the injection site. Alternatively, continuous vibration may be provided at the first contact site, and intermittent vibration provided at the injection site. Alternatively, intermittent vibration may be provided at the first contact site and at the injection site. The vibration is provided for a desired period, such as until the perception of pain is minimal. A medical provider or a user of the device can determine which type of vibration to use and how long to contact the device and/or the thermal element to the skin surface. Lidocaine also causes painful or irritating sensations (negative sensations) when injected, and injections of lidocaine may also be treated with a device of the present invention to relieve the painful and unpleasant sensations from providing the medication.

Methods of the present invention comprise reducing the negative sensations such as burning or itching caused by a medication injected into a subject, and such methods include medications that cause such sensations, not just those listed herein. For example, pharmaceutical or medical solutions infused into veins may cause pain to subjects receiving the solutions. Thought not wishing to be bound by any particular theory, it thought that the vibrational and/or thermal effects provided by a device of the present invention may block nerve transmissions from venous sensory nerves so that the burning and/or pain sensations from the infused medical or pharmaceutical solutions to the brain and/or spinal cord are blocked.

A method of the present invention comprises reducing an itching sensation in a subject, wherein the itching is localized to one or more areas. For example, the itching sensation may an acute reaction and be caused by an insect bite or an allergic reaction to contact with a substance or an injection which creates an irritated area of skin, or may be a chronic condition caused by constantly irritated nerves such as with dry skin, eczema or psoriasis, causing an irritated area of skin. A subject responds to itching by scratching, which may damage skin and lead to infection or permanent scarring of the skin. A method of the present invention comprises reducing scratching at a site of skin irritation, comprising a) contacting a device of the present invention with an irritated area of skin of a subject: b) initiating vibration by the device in an intermittent or continuous vibration, and optionally applying a thermal effect simultaneously with the vibration; c) continuing the vibration and/or thermal effect at the irritated skin site for a time sufficient to reduce the itching sensation felt from the irritated skin site. The thermal effect may cold or warm. The device may interfere with transmission of pain signals by aDelta nerves at the site of contact. The irritated skin is the source of the itching sensation. A method of the present invention comprises a method of reducing the itching sensation of a subject comprising, a) contacting a device of the present invention with an irritated area of skin of a subject; b) initiating vibration by the device in an intermittent or continuous vibration, and optionally applying a thermal effect simultaneously with the vibration; c) continuing the vibration and/or thermal effect at the irritated skin site for a time sufficient to reduce the itching sensation felt from the irritated skin site. The thermal effect may cold or warm. The device may interfere with transmission of pain signals by aDelta nerves at the site of the itching sensation. For example, a person has an area of skin that has eczema on her arm. A device of the present invention was kept by the bedside and when an itching sensation was triggered at the site of eczema of her arm in the night, instead of scratching the site and damaging the skin, the person contacted the device to the site of eczema on her arm, turned on the vibration effect for a period of 2 to 3 minutes. The device was then turned off, replaced on the bedside table, and the person returned to sleep. Use of the device prevented damage to the skin by scratching, and allowed for more restful sleep by the person.

A method of the present invention comprises treating pain from scrapes, such scrapes from falling on a rough surface, or from pain during medical cleaning or scraping of an open wound. When the structure of the skin is disturbed by a scrape, such as a rough abrasion of the skin due to a fall on a rough surface or rubbing against a surface such as in a motorcycle accident, many pain signals are sent to the brain. A method of interfering with transmission of pain signals caused by a scrape comprises a) contacting a device of the present invention with an area on the surface of a subject between the spinal cord and the site of the scrape, so that at least a portion of the application area of the device contacts the area; b) initiating vibration by the device in an intermittent or continuous vibration, and optionally applying a thermal effect simultaneously with the vibration, by interposing a thermal element between the application area of the device and the contacted surface; and c) providing vibrating and/or thermal effect for a sufficient time to interfere with nerve transmission to the brain, and to reduce the pain felt front the scrape site. The thermal effect may cold or warm. The device may interfere with transmission of pain signals by aDelta nerves at the site of the scrape. The device may be placed from 0.5 to 15 cm from the scrape site in a location that is between the scrape and the nerve plexus.

An open wound in the skin or underlying structures may be treated by medical personnel by scraping the wound with a blunt instrument to remove cellular debris or other debris present and this treatment is very painful, though necessary. A method of interfering with transmission of pain signals caused by a scraping treatment comprises a) contacting a device of the present invention with an area on the surface of a subject between the spinal cord and the site being treated by scraping, so that at least a portion of the application area of the device contacts the area; b) initiating vibration by the device in an intermittent or continuous vibration, and optionally applying a thermal effect simultaneously with the vibration, by interposing a thermal element between the application area of the device and the contacted surface; and c) continuing vibration and/or thermal effect at the site of for a time sufficient to reduce the pain felt from the site being scraped and treated. The thermal effect may cold or warm. The device may interfere with transmission of pain signals by aDelta nerves at the site of the wound or scraping treatment. The device may be placed from 0.5 to 15 cm from the scrape site in a location that is between the scrape and the nerve plexus.

A method of the present invention comprises treating neuropathic pain from nerve pain such as an outbreak of herpes simplex or shingles, or phantom pain from a missing limb. A method of interfering with transmission of neuropathic pain signals caused by viral infection such as cold sores or shingles, or from phantom limb pain, comprises a) contacting a device of the present invention with an area on the surface of a subject between the spinal cord and the site of nerve pain or in the case of missing limbs, between the spinal cord and the stump, or at the stump or terminus of the limb, so that at least a portion of the application area of the device contacts a portion of the surface area of the subject; b) initiating vibration by the device in an intermittent or continuous vibration, and optionally applying a thermal effect simultaneously with the vibration, by interposing a thermal element between the application area of the device and the contacted surface; and c) continuing vibration and/or thermal effect at the site of for a time sufficient to reduce the pain felt from the site. The thermal effect may cold or warm. The device may interfere with transmission of pain signals by aDelta nerves at the site. The device may be placed from 0.5 to 15 cm front the scrape site in a location that is between the scrape and the nerve plexus.

A burn site in the skin or underlying structures may be treated by medical personnel and burn treatment may be very painful, though necessary, and the healing period after a burn may be very painful even if no active treatment is made to the burn area. A method of interfering with transmission of pain signals caused by treatment of the burn or interfering with the transmission of pain signals from a burn when no active treatment is occurring comprises a) contacting a device of the present invention with an area on the surface of a subject between the spinal cord and the burn site, so that at least a portion of the application area of the device contacts the area; b) initiating vibration by the device in an intermittent or continuous vibration, and optionally applying a thermal effect simultaneously with the vibration, by interposing a thermal element between the application area of the device and the contacted surface; and c) continuing vibration and/or thermal effect at the site of for a time sufficient to reduce the pain felt from the burn site. The thermal effect may cold or warm. The device may interfere with transmission of pain signals by aDelta nerves at the burn site. The device may be placed from 0.5 to 15 cm from the burn site in a location that is between the burn and the nerve plexus.

Additionally, a burn site may have reduced blood flow into and/or out of the burn site. A method of increasing blood flow into and/or out of a burn site comprises a) contacting a device of the present invention with an area on the surface of a subject adjacent to the burn site, so that at least a portion of the application area of the device contacts the area: b) initiating vibration by the device in an intermittent or continuous vibration, and optionally applying a thermal effect simultaneously with the vibration, by interposing a thermal element between the application area of the device and the contacted surface; and c) continuing vibration and/or thermal effect at the site of for a time sufficient at least initiate increased blood flow into and/or out of the burn site. Multiple treatments of contacting an area adjacent to the burn site with a vibratory device of the present invention, optionally comprising a thermal element, may be desired to provide continued increased blood flow. The thermal effect may cold or warm. The device may provide vibratory effects including increasing blood or lymph flow. The device may be placed from 0.5 to 15 cm from the burn site.

Also disclosed herein are methods for using the disclosed for aiding in, enhancing or increasing healing of an injured site. The present invention comprises methods comprising use of a device disclosed herein for healing, such as, but not limited to, increasing biochemical and cellular responses that lead to healing of an injured site, such as increased granulation or skin production at a chronic wound or burn. A method disclosed herein comprises contacting a device disclosed herein to a site of injury in a subject, for example, to a chronic wound or burn, initiating vibration and/or thermal effects, for a predetermined time, such as a time sufficient to effect modulation of cellular and biochemical mechanisms such as angiogenesis, reduction of inflammatory cells, lowering the level of inflammatory cytokines, and increasing the level of pro-angiogenic factors and growth factors. For example, a device disclosed herein is provided to an area of the subject where the skin is injured, such as a burn or a chronic wound. For example, the device, comprising a thermal element, is placed on a chronic wound and is held in place. The device's switch is activated so as to turn on the vibrational source, and the thermal and vibrational effects are transmitted to the acute or chronic injury or wound area. This treatment may be repeated hourly or daily or for any length of time to modulate cellular and biochemical mechanisms such as angiogenesis, reduction of inflammatory cells, lowering the level of inflammatory cytokines, and increasing the level of pro-angiogenic factors and growth factors. The treatment may continue for several minutes at a time, wherein the vibration and thermal effects are provided by the device to the chronic wound, and such treatments may be repeated one or more times a day for one or more days, weeks, or months. With repeated treatment, the extent of the injury is reduced and the injured site may be resolved. With reduction in injury, pain is also reduced.

In further aspects, treatment times of the disclosed methods may comprise from about 0.5 minutes to about 2 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 1 minute to about 60 minutes, from about 1 minute to about 90 minutes, from about 1 minute to about 100 minutes, from about 1 minute to about 120 minutes, from about 20 minutes to about 30 minutes, from about 20 minutes to about 60 minutes, from about 20 minutes to about 90 minutes, from about 20 minutes to about 120 minutes, and ranges therebetween. The treatment times listed here are not contemplated to be limiting to the methods of treatment of the invention. One of skill in the art can determine optimal treatment time periods.

In various aspects, methods for using the disclosed devices include, but are not limited to, methods to control pain associated with injections, venipuncture, IV starts, cosmetic injections (e.g., Botox injections), temporary relief of minor injuries (muscle or tendon aches, splinters and bee stings), and treatment of myofascial pain caused by trigger points, restricted motion and muscle tension. Wound healing and pain relief treatment methods are also accomplished by use of a device disclosed herein to provide thermal and/or vibratory effects to one or more sites on the subject.

The thermal element is cooled or heated, if necessary to within a predetermined temperature range. The thermal element may be placed within or attached to the casing. Alternatively, if the device is made to certain standards, the entire device already containing a thermal element can be cooled to the desired temperature. When a subject anticipates a need for a treatment using the device, the device is applied to the body at a desired location, such at the site to be treated or at a site proximate to a pain site and between the pain site and the brain and/or spinal cord, as described for treatments disclosed herein.

When a subject is to undergo a medical procedure, such as infusion of a medicinal solution that causes a burning or painful sensation, or scraping of a wound, or when deemed necessary by a subject, a device of the present invention comprising a thermal element may be applied to a selected area of the subject such that the vibrational area contacts the subject's skin through the thermal element to provide vibrational and thermal effects to the subject, for example, through the application area. The optional thermal element may be allowed to act upon the subject for a time necessary to initiate thermal effects, which can be for a period of seconds up to a period of several minutes or hours.

In further aspects, thermal treatment times of the thermal effects may comprise from about 0.5 minutes to about 1 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 1 minute to about 60 minutes, from about 1 minute to about 90 minutes, from about 1 minute to about 100 minutes, from about 1 minute to about 120 minutes, from about 20 minutes to about 30 minutes, from about 20 minutes to about 60 minutes, from about 20 minutes to about 90 minutes, from about 20 minutes to about 120 minutes, and ranges therebetween. The treatment times listed here are not contemplated to be limiting to the methods of treatment of the invention. One of skill in the art can determine optimal thermal treatment time periods.

Once suitable thermal effects are achieved, or concurrently when the thermal element is applied to the subject, the vibrational source is actuated by the on/off switch, creating vibration. The vibrational source is allowed to act upon the subject for a time necessary to initiate vibrational effects which can be for a period of seconds up to a period of several minutes or more, or may be from about 0 to about 60 seconds. If prolonged vibratory and/or thermal treatment is desired the device may be applied for a longer period to provide pain relief or relief from unpleasant sensations. Once the desired outcome is reached, the device may be removed from the subject, and/or the thermal element can be removed from acting on the subject and/or the vibrational source can be turned off. However, it is possible to leave the device, including the active thermal element and the active vibrational source in contact with the subject for prolonged periods of time. For example, the device may be left in place by using a wrap, and the device is then activated on an on-going schedule of time periods of use of the device and quiescence. One or more thermal elements may be provided to the device to allow for thermal effects to the subject during the periods of use.

“Thermal effects” as used herein includes, but is not limited to, the use or application of cold or reduced temperature (or the removal of heat) thermal elements or use of warm or heated thermal elements to a subject to induce a thermal effect in the subject, such as, for example, increased vascular diameter and increased arterial or venous blood flow, or constriction of vessels or inhibition of pain transmission.

“Vibrational effects” as used herein includes, but is not limited to, the use or application of vibration to a subject to induce vibrational responses in the subject, such as an increase in vascular diameter and increased blood flow from arteries, veins, or capillaries, or blocking of pain transmission by nerves, such as aDelta nerves, or blocking or reducing of burning, itching or other unpleasant sensation transmission to the brain and/or spinal cord. Vibrational effects also include stimulation of the subject, in that vibration provides a stimulatory effect to contacted tissues or nerves, and may also stimulate adjacent or more distant sites of the subject.

“Vibrational and thermal effects” as used herein includes, but is not limited to, the use or application of either heat or cold or reduced temperature (or the removal of heat) concurrently, substantially concurrently, or sequentially with the use or application of vibration to a subject to induce physiological changes in the subject in the area contacted by a device of the present invention or in a proximal or distal area.

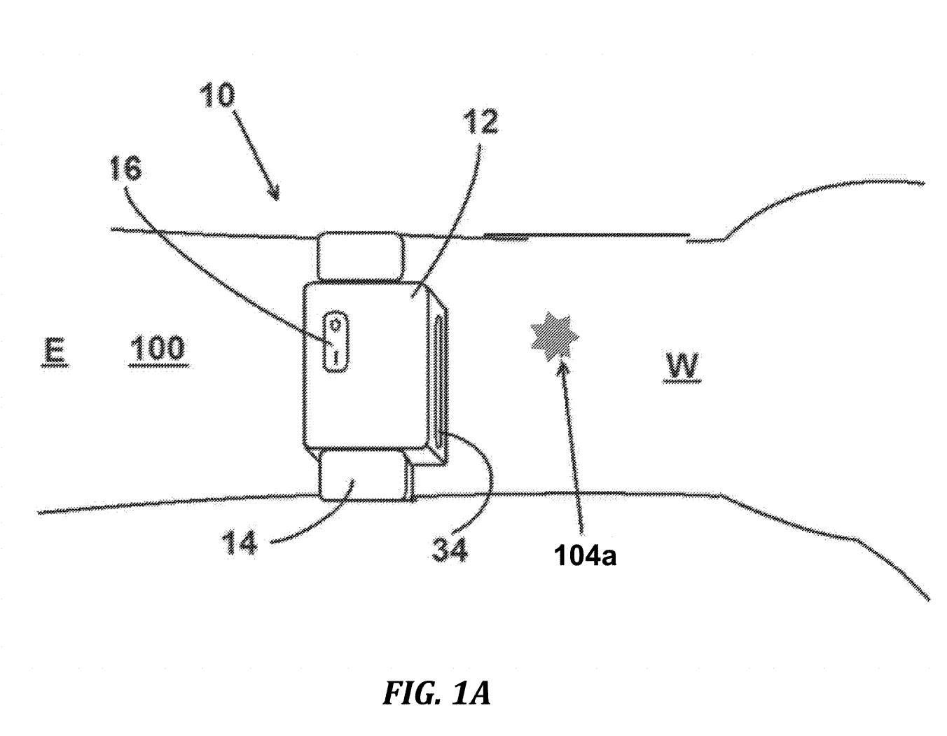

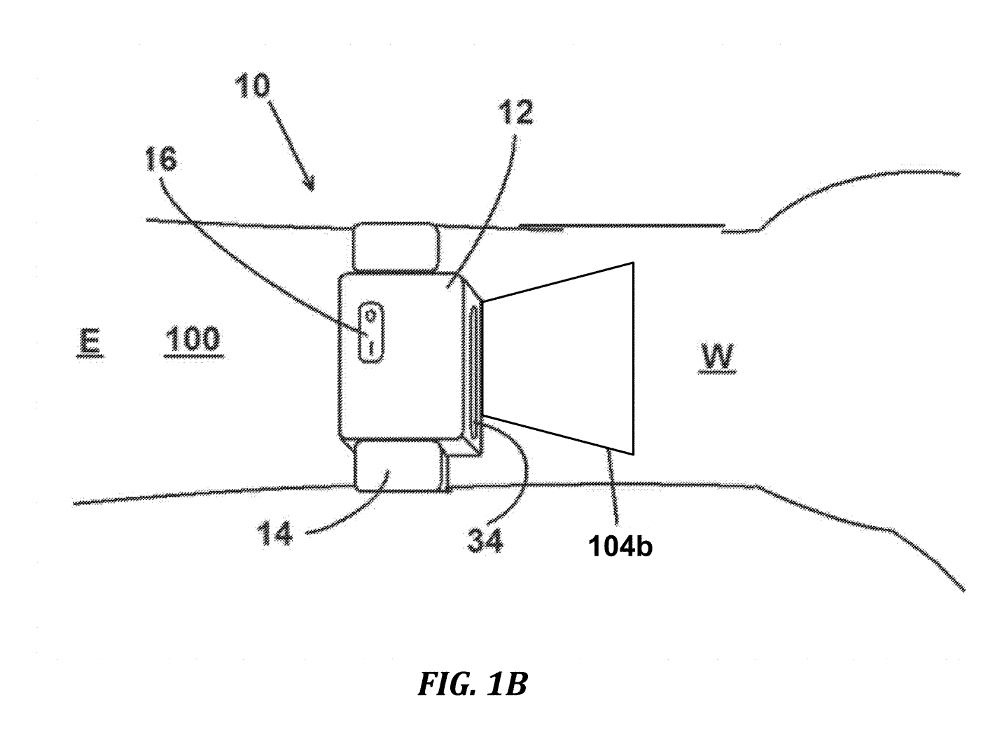

Referring now to FIGS. 1A-1B, perspective views of an embodiment of the device is shown as applied to the arm of a subject at a site, showing the casing that houses the various components of the invention and an optional strap for holding the device to the subject. In the FIG. 1A view, the device (10) is being applied to the surface (100) of a subject who has an initiation site (104a), which may be a site of pain or irritating sensation, such as burning or itching. In the FIG. 1B view, the device (10) is being applied to the surface (100) of a subject who has an injury site (104b), which may a site of a wound or injury. As used herein, an initiation site is a site on a body that has reduced blood flow, reduced lymph flow, blocked ducts, is a site for painful sensations, a site of neuropathic pain, a site for unpleasant sensations including, but not limited to, itching and burning sensations, and those from insect stings, scrapes, or other abrasions to a body surface. For example, the initiation site may have reduced blood flow and contacting the initiation site with a vibrating and/or thermal effects providing device of the present invention may increase the blood flow or cause local vasodilation. For example, the initiation site may be a wound or injury site, and contacting a site other than the initiation site, such as between the initiation site and the brain and/or spinal cord of a subject with a vibrating and/or thermal effects providing device of the present invention may reduce, impede or prevent transmissions of painful stimuli sensations to the brain.

In some aspects, the positioning of the device (10) on the subject is between the initiation site (104a) and a nerve plexi (not shown, but the location of which is known to those of ordinary skill in the medical, field) such as between the initiation site (104a) and the proximal joint, in this figure, an elbow (E). Thus, for the illustrated initiation site (104a) on a subject's arm (100), the device (10) is placed closer to the elbow (F) than to the wrist (W) as this location puts the device (10) in between the initiation site (104a) and the subject's brain. For example, the device 10 may be placed approximately 2.5 cm to 15 cm from initiation site (104a). The on/off switch (16) is shown on the distal side of the device or casing, and a strap (14) is shown holding the device on the subject. A cuff, wrap, bandage or other similar component can be used in place of a strap to hold a device of the present invention on a subject.

In further aspects, a casing (12) may be manufactured of a flexible or pliant material such as for illustrative purposes a natural or synthetic woven or non-woven fabric, a rubber or other flexible polymer material, a silicone-based material, or may be a rigid material, such as a plastic, metal or wooden casing, wherein the casing is a container with walls to define an enclosed area. Other flexible or pliant or other materials may be employed, and the material of construction may be non-toxic, hypo-allergenic and non-staining to the subject. A material that will transfer vibrations and/or thermal effects is contemplated by the present invention. In still further aspects, the material may comprise both excellent thermal conductivity and mechanical performance properties. In yet further aspects, the material may comprise thermally conductive material such as metals or thermally-conductive thermoplastic compositions. Examples of suitable metals include, but are not limited to, aluminum, copper, steel, and bronze, or combinations thereof. Thermally conductive, thermoplastic compositions may comprise one or more polymers and one or more thermally conductive fillers. Examples of suitable thermally conductive filler include, but are not limited to, AlN (Aluminum nitride), Al4C3 (Aluminum carbide), Al2O3 (Aluminum oxide), BN (Boron nitride), AlON (Aluminum oxynitride), MgSiN2 (Magnesium silicon nitride), SiC (Silicon carbide), Si3N4 (Silicon nitride), graphite, expanded graphite, graphene, and carbon fiber, or combinations thereof. In even further aspects, different portions of the casing may comprise different materials. In some aspects, a main housing of the casing containing the vibrational source may comprise a first material having good vibrational transfer characteristics in order to effectively transfer vibrations, and a separate compartment of the casing, such as a thermal element pocket containing a thermal element, may comprise a second material having both excellent thermal conductivity and mechanical performance properties in order to effectively transfer both vibration and thermal effects, for example, thermal effects from thermal element as well as vibrations transmitted through the thermal element and thermal element pocket from the vibration source contained in the main housing.

The casing can be any shape, and preferably is in the shape of a three-dimensional polygon (for use with an adult use) or an animal or other distractive shape (for use with a child) and the casing walls define an interior space or interior sections for containing the operating elements of the invention. Any other shape (as used herein, the term shape is used in the broad sense of three-dimensional works) may be employed, so long as the shape is large enough and structured so as to be able to contain the various working components of the invention as more fully disclosed below.

In further aspects, an exemplary embodiment of the device is shown in FIGS. 1A-1B applied to the arm of a subject having an initiation site (104a), which may be a painful site, such as a burn, a scrape and viral outbreak, or a site of medication infusion that is painful when infused, or may be a site of itching or other unpleasant and undesired sensation; and applied to an injury site (104b), which may be a wound site. The positioning of the device on the subject may be between the initiation site (104a) and a nerve plexi (not shown, but the location of which is known to those of ordinary skill in the medical field) or between initiation site (104a) and the spinal cord or brain. In this FIG. 1A example, the device is placed at or proximal to the elbow E. In some aspects, the device may be placed approximately 2.5 cm to 15 cm from the initiation site (104a). In other aspects, the device may be placed approximately 0.1 cm to 55 cm from an injury site (104b).

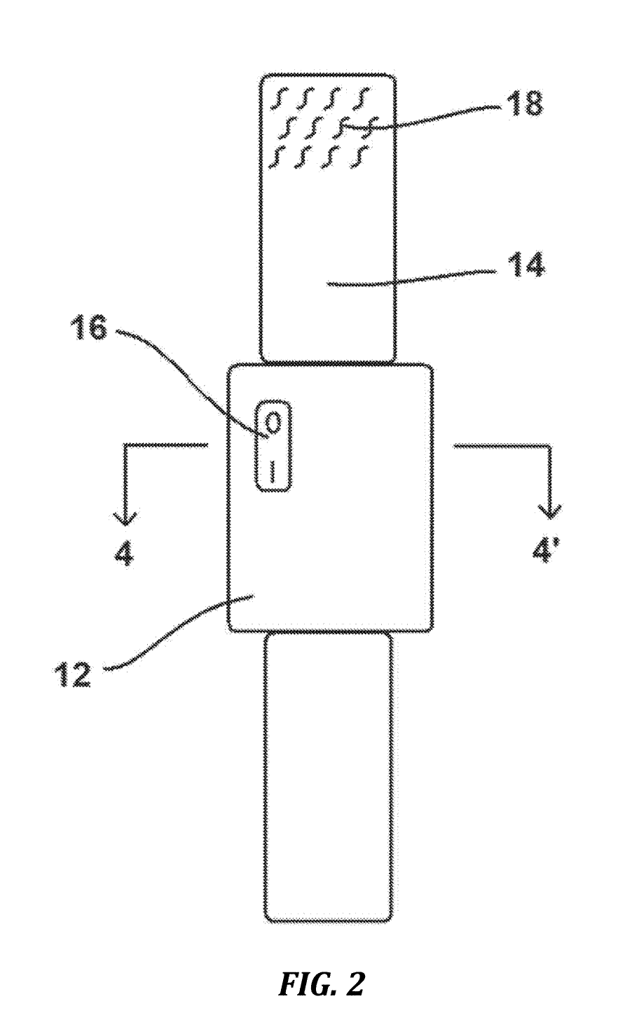

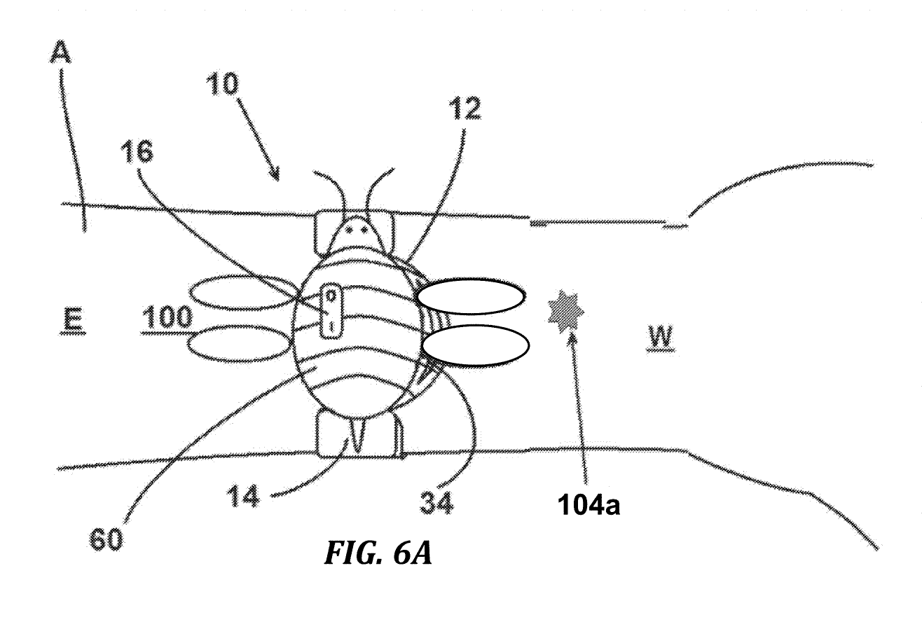

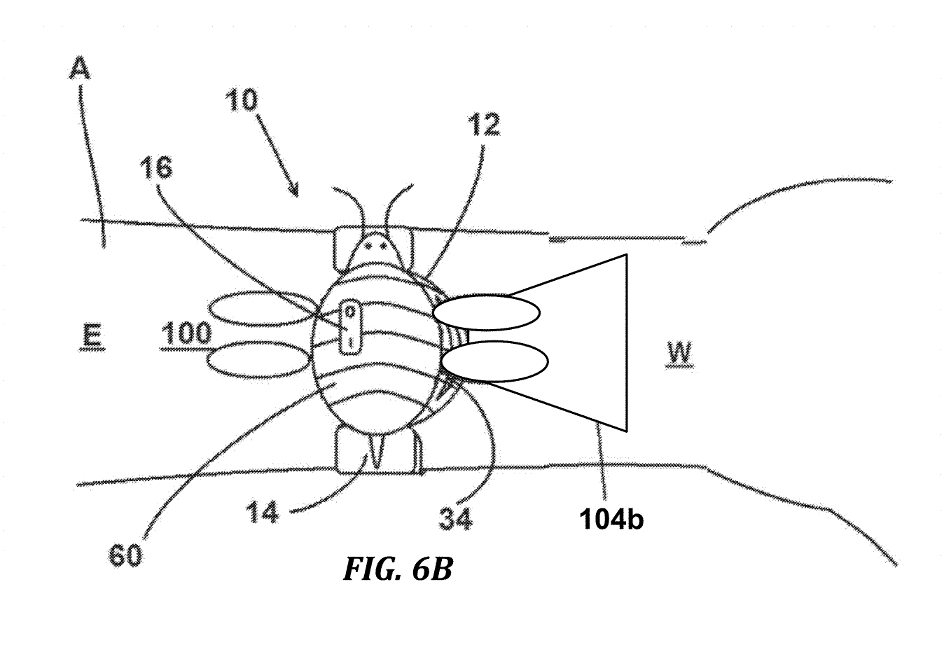

In further aspects, a thermal element pocket (34) in the casing is illustrated on this embodiment. An embodiment showing the distal side of the invention of FIGS. 1A-1B is shown in FIG. 2, comprising the casing (12) and an on/off switch (16). An optional strap (14) can be used to hold the device on to the subject. Alternatively, the device can be held against the subject by medical personnel, or the subject. The strap (14) can be attached to the casing (12) in any conventional manner or can be an extension of casing itself. For example, the strap and casing can be attached together much like a conventional watch and watchband with hinges or pins. Or in another embodiment, the strap can be an extension of the fabric or other material enclosing the casing, such as an extension of a decorative cover (shown in FIGS. 6A-6B). The ends of strap preferably have some type of connection element (18), such as a hook and loop fastener, adhesive, a clasp, a clip, snaps, magnets, or the like for attaching the device about the subject's body pan. Alternatively, if the ends of the strap are flexible, the ends can be tied together around the subject's body part. In some aspects, the strap can be a continuous band, with both ends attached to the casing or cover.

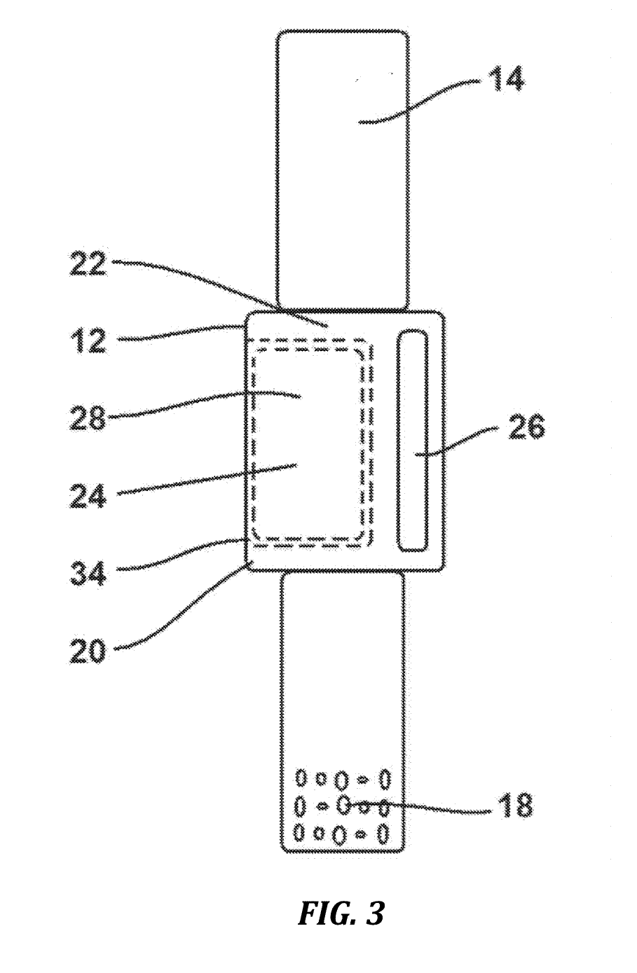

Referring now to FIG. 3, a bottom or proximal view of an exemplary embodiment of the invention is shown. The casing (12) has a peripheral bottom rim that defines an application area (22). Application area (22) comprises thermal area (24) and vibration area (26). Although thermal area (24) and vibration area (26) area shown as discrete areas, this is for illustrative purposes only, as there need not be any physical delineation between thermal area (24), vibration area (26), and application area (22) and these areas can overlap and be coexistent as the same area, which may be referred to as application area (22). Thermal element (28) cooperates with thermal area (24) to apply cold or heat to the subject, and vibrational source (32) (not shown) cooperates with vibration area (26) to apply vibration to the subject. Thermal area (24) and vibrational area (26) can occupy the same area, or can coextend and form application area (22). Thermal element (28) may be located within thermal element pocket (34). The thermal pocket (34) is a slot, fold or other type of compartment in the casing into which the thermal element can be placed. As shown in FIG. 3, the thermal element pocket is accessed on the side of the casing via a mouth or an opening in the casing. Alternatively, the opening for the pocket can be located at other sites on the casing depending on the size and shape of casing and the location of the vibrational source within the casing. Alternatively, the thermal element can be contained within the main housing volume of the casing. Thus, the placement of the thermal element is variable so long as the cooling or heating effects of the thermal element can be felt on the subject so as to produce thermal effects. Thermal area in its simplest form is an area on the application area on the device that allows the thermal effects from thermal element to contact the subject.

In further aspects, the vibration area can comprise an area on the casing in vibratory contact with the vibrational source. As disclosed in more detail below, vibrational source preferably is contained within the main housing volume of the casing. The placement of the vibrational source is variable so long as the vibration effects of vibrational source can be felt on the subject so as to produce vibrational effects. As shown in FIG. 3, vibrational area is proximal to thermal area; however, vibrational area can coextend with thermal area. In still further aspects, vibrational area in its simplest form is an area on the application area on the device that allows the vibrations from vibrational source to contact the subject.

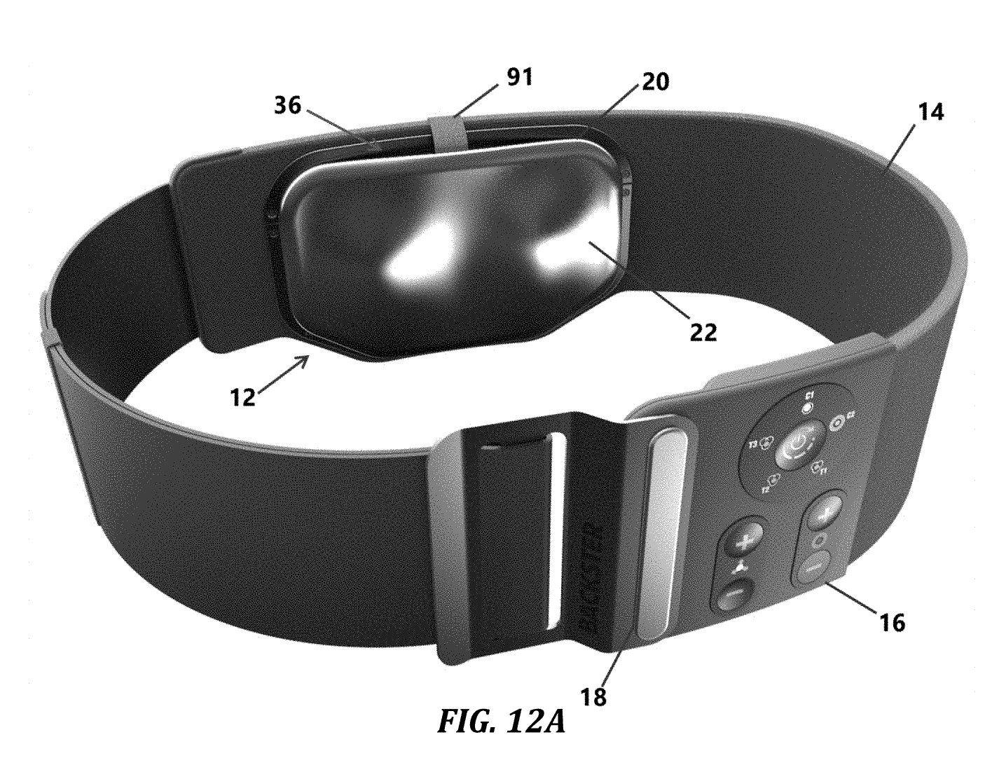

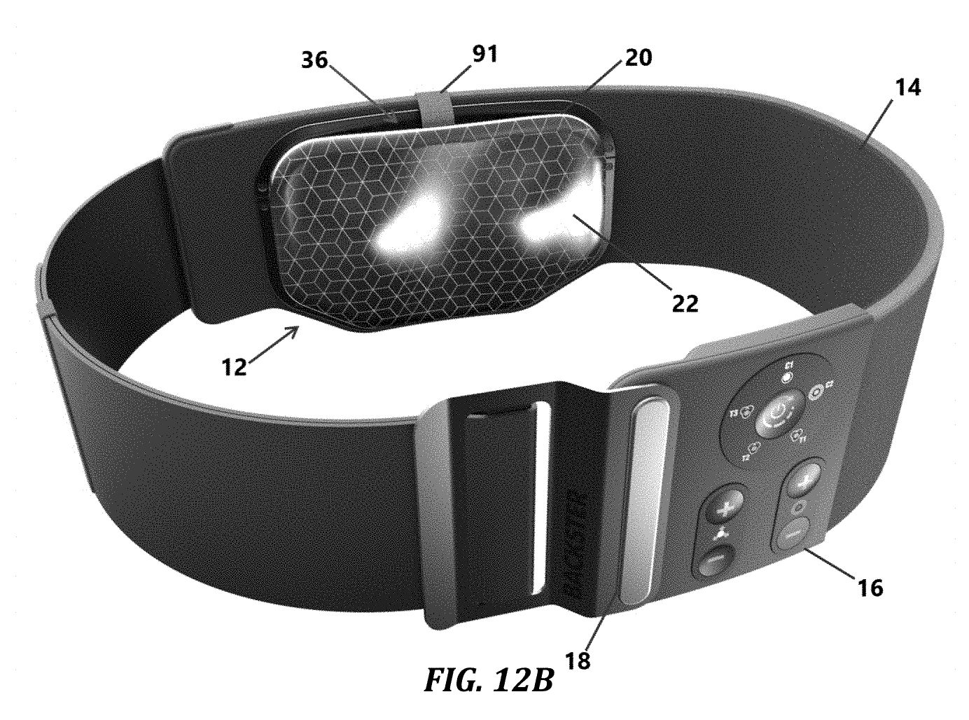

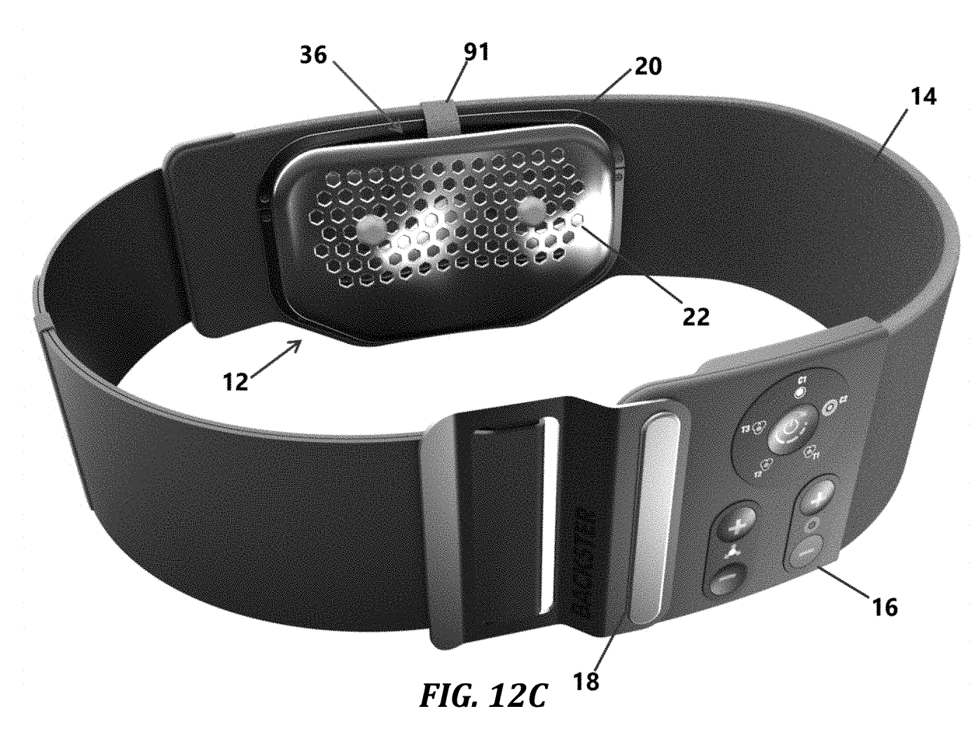

In still further aspects, FIGS. 12A-12C show an exemplary embodiment of external features of the invention comprising a casing (12) with strap (14) for holding the vibratory device on to the subject and a remote switch (16) for controlling operation of the vibratory device. Strap (14) can be an extension of casing (12) itself or can be attached to the casing (12) in any conventional manner. The ends of strap (14) may have some type of connecting device (18), such as a clasp, a ring, a hook and loop fastener, a clip, snaps, magnets, adhesive, or the like for attaching the device about the subject's body part. The remote switch (16) may be located at the end of strap (14), and the connecting device (18) may connect to a portion of the remote switch (16). The casing (12) may have a peripheral rim (20) that defines an application area generally designated (22). The casing (12) may be curved, such as the convex areas shown in application area (22), in a manner to enhance proximity and, thus contact, of the device by a strap or wrap. The application area (22), can take the form of a solid plate (FIG. 12A and FIG. 12B) or can further comprise apertures for receiving nodes for targeted therapy to a designated area of a site (FIG. 12C).

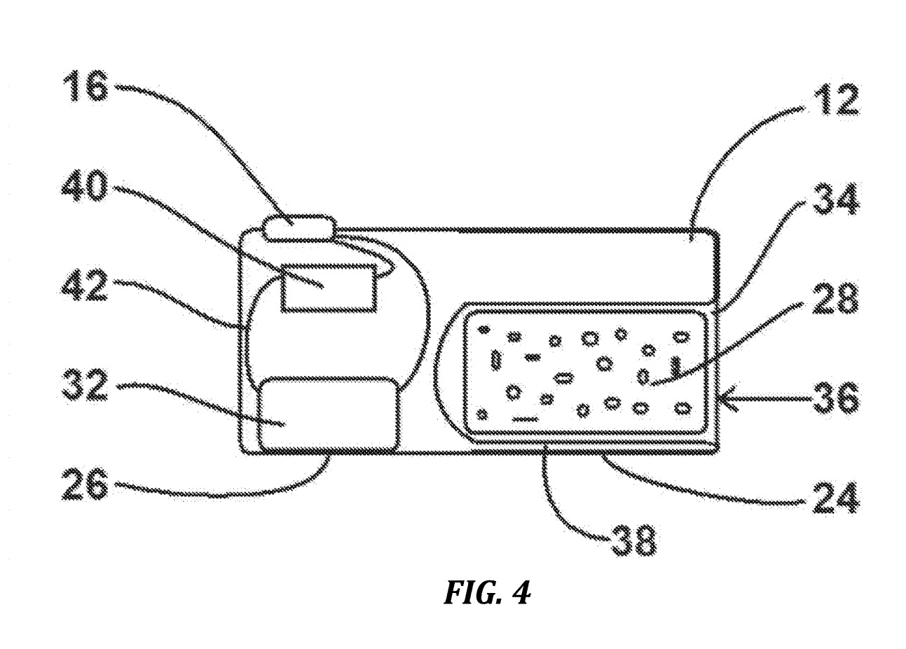

Referring now to FIG. 4, a sectional side view of the embodiment of the invention as shown along line 4-4′ of FIG. 2 is shown. The casing (12) is a generally hollow structure sized to contain an optional thermal element (28), and at least one vibrational source. A casing (12) can be a rigid hollow container having an interior volume or a flexible or pliant container having an interior volume. Such containers are known, as well as their materials and methods of construction are within the skill of those in the art. The casing may be constructed such that casing (12) can contain and hold a vibrational source, and optionally, a thermal element or source, in a predetermined position relative to the subject when the device is contacting a subject.

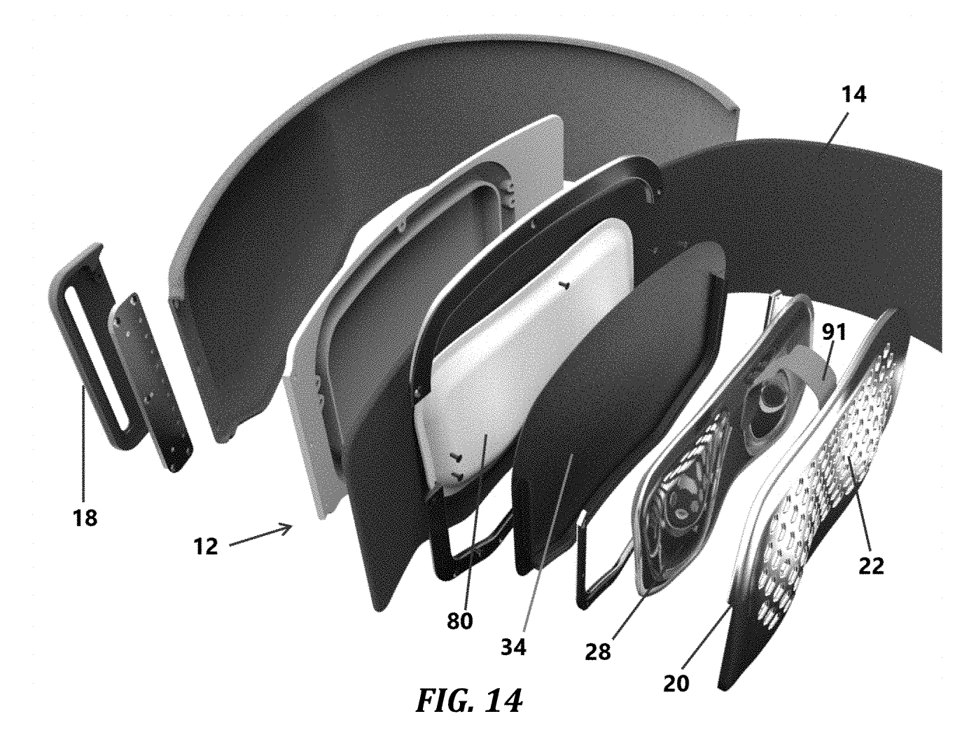

As shown in FIG. 14, thermal element (28) can be placed within a thermal element pocket and can be held within thermal element pocket (34) by closure (91) or friction, adhesives, fasteners, or other type of closure. Vibration source may be disposed on an interior surface of application area (22) or otherwise located between the thermal element (28) and application area (22). To this end, thermal element (28) and vibrational source can cooperate with application area (22) to apply vibration in conjunction with cold or heat to the subject. In other aspects, the casing (12) can further comprise an inflatable air bladder (80). The air bladder (80) can be filled or emptied to modulate the level of thermal effect and/or vibration effect transmitted through the application area (22) to the user.

As shown illustratively in FIG. 4, thermal element is contained in thermal element pocket. Thermal element can be placed within thermal element pocket through mouth or opening and can be held within thermal element pocket by friction, adhesives, fasteners, or by a zipper or other type of closure on the pocket mouth or opening. The proximal side of a casing may comprise the bottom or outer wall of thermal element pocket. It may be desired that the bottom or outer wall of thermal element pocket may be sufficiently thin or have sufficient thermal transfer characteristics so as to allow the efficient transfer of cold or heat from thermal element to the subject. Further, the bottom or outer wall of thermal element pocket may have sufficient vibrational transfer characteristics so as to allow the efficient transfer of any vibration from the vibrational source, for example, that may be transmitted through the thermal element to the subject. The wall of application area (22) may be comprised of a material having both sufficient vibration transfer and thermal transfer characteristics (e.g., metal) so as to allow the efficient transfer of both vibration and thermal effects to the subject through the application area (22). In some aspects, a thermal element (28) may be placed on the outside surface of the proximal side of a casing so that in use, the thermal element is interposed between the outside proximal surface of the casing and the subject's surface. In other aspects, a thermal element (28) may be placed on the inside surface of the proximal side of a casing so that in use, the thermal element is thermal communication with the interior proximal surface and nothing is interposed between the exterior proximal surface of the casing and the subject's surface such as to allow maximum transfer of vibration and/or thermal effects to the subject's surface.

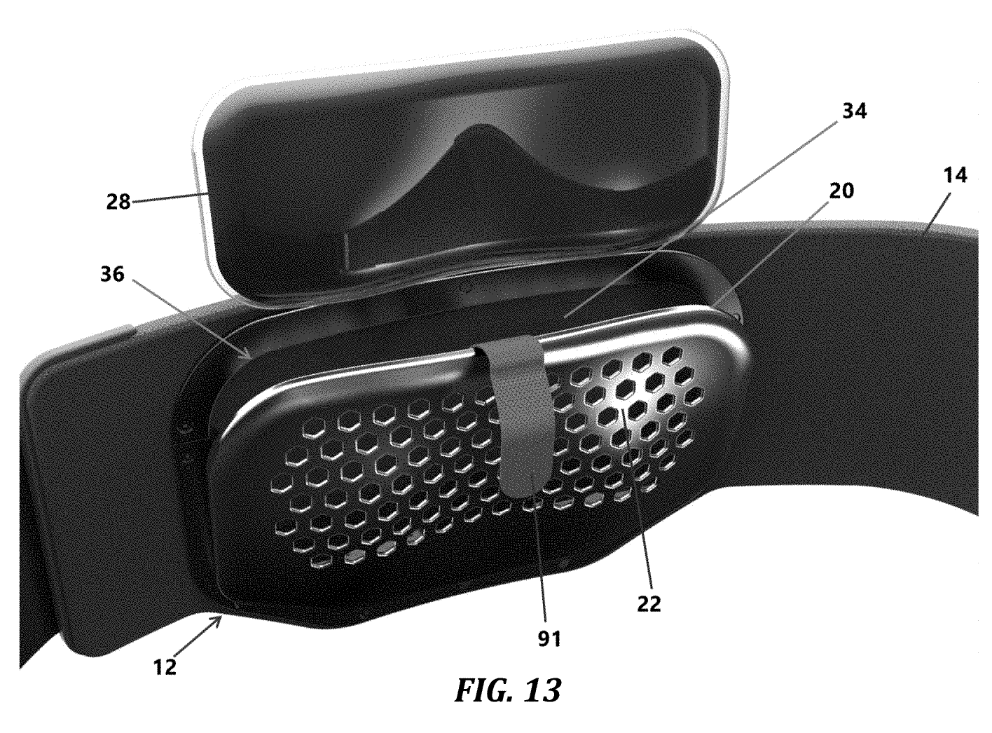

As shown in FIG. 13, all or a portion of an interior surface of application area (22) may contact or otherwise be in communication with a thermal element while all or a portion of an exterior surface of application area (22) may contact the subject. The application area (22) comprises at least one of: thermal area and vibration area. Although in the present embodiment the thermal area and vibration area are shown overlapping and coexistent as the same area, these areas may be discrete with physical delineation between these areas as further described herein. Thermal element (28) can be placed within thermal element pocket through mouth or opening (36) and can be held within thermal element pocket (34) by friction, adhesives, fasteners, or by a zipper or other type of closure (91) on mouth or opening (36). In some aspects, a thermal element (28) within thermal element pocket (34) may be in thermal communication with application area (22) and a vibrational source (which may be disposed on an interior surface of application area), thus allowing application area (22) to cooperatively transmit both vibration and thermal effects to the subject.

In some aspects, thermal element can be any conventional thermal element capable of storing and transferring heat or cold (or removing heat). Illustrative examples of suitable thermal elements include metal ingots, low freezing point (below about 45° F. or 7.2° C.) liquids and gels, ceramics, clays, polymers, polymer materials, natural materials such as bran, other heat sinks, hot packs, chemical reactive thermal packs, thermal gel packs, thermal clay packs, and even ice packs. Such thermal elements are known. It is only important that thermal element be able to transfer cold or heat to the subject in a sufficient amount so as to produce the desired effect, for example vasodilation, pain reduction, itching sensation reduction, or reduction in blocked vessels. For example, providing a temperature of below about 55° F. or 12.8° C., and between about 15° F. or −9.4° C. and about 55° F. or 12.8° C., or between about 28° F. or −2.2° C. and about 50° F. or 10° C. or between about 38° F. or 3.3° C. and about 45° F. or 7.2° C., or for example about 24° F. or 34° F. to the subject prior to and during the treatment method is sufficient to provide a suitable level of effective thermal treatment. In other aspects, the temperature may be above about 95° F. or 35° C., and between about 100° F. or 37.8° C. and about 120° F. or 48.9° C. or between about 105° F. or 40.6° C. and about 115° F. or 46.1° C., or for example about 110° F. or 43.3° C. to the subject prior to and during the treatment method is sufficient to provide a suitable level of effective thermal heat treatment.

In further aspects, the thermal element is applied to the subject for a time period sufficient to initiate treatment, such as thermal vasodilation, which can be between 0 seconds and several minutes or more depending on the subject. For example, in some applications, it is desirable to apply the thermal element to the subject for a period of about 0 to 60 seconds, or longer, prior to initiating an activity, such as injecting a medication that causes a painful or burning sensation, or scraping of a wound, and continuing the application of the device may provide a thermal effect and/or vibration, for example, during the activity, to provide a suitable level of effective treatment, by the device.

In other aspects, the thermal element may be any conventional thermal element capable of storing and transferring heat or cold. Illustrative examples of suitable thermal elements include high specific-heat capacity material like grains, such as wheat or buck wheat, sewn within an insulated fabric such as flannel, chemical thermal elements like calcium chloride- or supersaturated sodium acetate-based heat pads, phase change materials, or other conventional heat/cold packs. A thermal element may be a gel or other type of heat/cold pack that may be placed in a freezer or microwave and such heat/cold packs are known in the art. The present invention contemplates use of thermal elements that are known in the art. The thermal element needs to transfer heat or cold to the subject in a sufficient amount so as to produce the desired effect of such heat or cold, for example vasoconstriction or vasodilation. The thermal element may be comprised of a material having mechanical performance properties that will effectively transfer vibrations to produce the desired vibration effect, for example, in embodiments where vibrations are transmitted from the vibrational source through the thermal element to the subject.

One of skill in the art, such as medical personnel, or a subject, can determine an adequate temperature and time for application of the thermal element for methods disclosed herein. The thermal element is applied to the subject for a time period sufficient to cause the desired effect, which can be between 0 seconds and several minutes or more depending on the subject and/or the method. A second or third thermal element may be used in replacing a first thermal element used in a method, especially in methods where application of vibration and/or thermal effects continue for a longer time period than the first thermal element can maintain the desired temperature.

In further aspects, the thermal element may comprise a powered or electric thermal element, such as, for example, an electric heating element and/or thermoelectric device. In still further aspects, an electric heating element may comprise at least one of: metal heating elements, ceramic heating elements, polymer positive temperature coefficient (PTC) heating elements, and composite heating elements. Examples of suitable metal heating elements include, but are not limited to, wire, ribbon, or strips made from nichrome (80% nickel, 20% chromium), kanthal (FeCrAl), and cupronickel (CuNi). In yet further aspects, a thermoelectric device can comprise a Peltier cooler, a Peltier heater, thermoelectric heat pump, or thermoelectric cooler, or combinations thereof. In some embodiments, the thermoelectric device may comprise two sides, and when a DC electric current flows through the thermoelectric device, it brings heat from one side to the other, so that one side gets cooler while the other gets hotter. The “hot” side may be attached to a heat sink so that it remains at ambient temperature, while the cool side goes below room temperature. In other embodiments, multiple thermoelectric devices can be connected together to achieve lower temperatures. In further aspects, the disclosed device may comprise a plurality of thermoelectric devices positioned with opposing electrical circuits, wherein a first portion of the plurality of thermoelectric device have the “hot” side on a first surface of the device or device casing and a second portion side of the plurality of thermoelectric devices have the “hot” side on a second or opposed surface of the device or device casing.

In further aspects, the first or second portion of the plurality of thermoelectric devices may be selectively activated to deliver heat or cold thermal effects to the subject. In yet further aspects, a predetermined number or portion of the first or second portion of the plurality of thermoelectric devices may be selectively activated to deliver a corresponding level of heat or cold thermal effects to the subject. By way of non-limiting example, about 25% of the first or second portion of the plurality of thermoelectric devices may be selectively activated to deliver about 25% of the maximum level of corresponding heat or cold thermal effects to the subject, about 50% of the first or second portion of the plurality of thermoelectric devices may be selectively activated to deliver about 50% of the maximum level of corresponding heat or cold thermal effects to the subject, or about 75% of the first or second portion of the plurality of thermoelectric devices may be selectively activated to deliver 75% of the maximum level of corresponding heat or cold thermal effects to the subject. In some aspects, one or more thermal elements may be disposed on the proximal surface (e.g., outermost) of the device or casing. In other aspects, one or more thermal elements may be disposed on or in thermal communication with the surface opposed to the proximal surface (e.g., outermost) of the device or casing wall. To this end, the thermal effects can be conducted through the device or casing wall to the proximal surface, and to the surface in contact with the proximal surface.

As shown illustratively in FIG. 4, the vibrational source (32) is contained within the interior of the casing (12). Vibrational source (32) can be placed within casing (12) during manufacture or at any time after manufacture. An ingress and egress element (90) is preferred, as one embodiment of vibrational source utilizes a battery as the power source, and it may be necessary to change the battery on occasion. For example, FIG. 9C where an ingress/egress element (90), as illustrated, is a screw, is shown for opening the casing (12), and may be used for holding a control board, and/or power source and/or vibrational source (motor), or other components on a control board in the interior of the casing (12). Ingress/egress element (90) can be a snap, a screw, a bolt, or any closure components that would releasably hold the casing closed and allow for access to at least a power source within the casing. Vibrational source (32) and power source (40) can be held within casing by friction, adhesives, fasteners, or other types of securing means. Alternatively, the interior volume of casing can be approximately the same dimensions as the vibrational source, including the power source, such that additional means for securing the vibrational source 28 are unnecessary. In aspects, the proximal side (30) of the casing which is adjacent to vibrational source be sufficiently thin or have sufficient vibrational transfer characteristics so as to allow the efficient transfer of vibration from vibrational source to the application area (22) of the casing (12) and thus to the subject to be treated in the methods disclosed herein.

In further aspects, vibrational source (32) can be any conventional vibrational source or means for producing vibrations. In even further aspects, device may comprise a plurality of vibration sources. The plurality of vibration sources may comprise at least two vibration sources, for example, two, three, four, five, or six vibration sources, or more. As shown in FIG. 4, vibrational source further comprises a power source (40) and wiring electrically connecting vibrational source and power source to an on/off switch. Illustrative examples of suitable vibrational sources include elliptical flywheel motors, eccentric motors, and the like. Such vibrational sources are known. It is only important that the vibrational source be able to transfer vibration to the subject at a sufficient level to produce the effect intended in the disclosed methods. For example, a device of the present invention can provide vibrations of between about 175-250 Hz. The application area of the device which vibrates due to the action of the vibrational source is applied to the subject for a time period sufficient to accomplish the effect intended in the disclosed methods, which can be between 0 seconds and several minutes or more depending on the subject and/or the method. For example, the application area of the casing may provide vibration to the subject for a period of about 0 seconds to about 60 seconds, or longer in certain methods, to accomplish the effect intended in the disclosed methods. A vibrational source may be a high frequency low amplitude eccentric motor. The motor may be controlled by a logic control board, such as a polycarbonate board, which known in the art. The motor and/or a power source may be held to a board by brackets, screws or other known attachment elements.

In further aspects, the disclosed devices can be configured to deliver vibrational and/or electrical stimulation. In some aspects, the vibrational source can be further configured to produce electrical stimulations to a surface of the administration site. In other aspects, the device can further comprise one or more electrical source configured to deliver an effective amount of electrical current to stimulate a site or nerves of the patient. In further aspect, the electrical source may comprise a transcutaneous electrical nerve stimulation (TENS) device configured to produce and deliver electric current to stimulate the nerves. In still further aspects, the electrical stimulation can comprise any range of transcutaneously applied currents used for nerve excitation. In still further aspects, the device may comprise one or more electrodes disposed on an outer surface of the casing configured to contact a surface, such as skin, of the subject. In yet further aspects, the device may be configured to modulate pulse width, frequency and intensity, such as, for example, at high frequency (>50 Hz) with an intensity below motor contraction (sensory intensity) or low frequency (<10 Hz) with an intensity that produces motor contraction. In various aspects, the electrical stimulation may be utilized in any configuration of the disclosed devices and methods. In some aspects, the disclosed devices may deliver vibration, electrical, and thermal stimulation concurrently to a site or surface of a subject. For example, in embodiments where electrodes or an electrical stimulation application area is configured as a portion of the casing in contact with the patient, vibration from the vibrational source may have transmission path beginning at the vibrational source and traveling through the vibration area, the thermal element (and optionally the attachment element), and electrical stimulation application area before reaching the administration site of the subject. Thus, in such embodiments, the device would be able to concurrently deliver vibration, electrical stimulation and thermal effects to a subject.

In further aspects, vibration treatment times may comprise from about 0.5 minutes to about 1 hour or longer, from about 1 minute to about 5 minutes, from about 5 minutes to about 15 minutes, from about 5 minutes to about 20 minutes, from about 10 minutes to about 20 minutes, from about 5 minutes to about 30 minutes, from about 1 minute to about 40 minutes, from about 1 minute to about 50 minutes, from about 20 minutes to about 30 minutes, from about 1 to 2 hours, from about 1 to about 3 hours, from about 2 to about 4 hours, and ranges therebetween. The treatment times listed here are not contemplated to be limiting to the methods of treatment of the invention. One of skill in the art can determine optimal vibrational treatment time periods.

In various aspects, the vibration level or intensity of a vibration source or element may be defined by reference to the amplitude and the frequency of the vibrations of the vibration source or element. To this end, the amplitude of a vibration of a vibration source or element is characterized as the strength or power of the vibration that the user feels when in contact with the vibration. In further aspects, high amplitude vibrations may be strong vibrations that a user feels are more powerful or forceful against his or her body than a weak vibration having a low amplitude. In still further aspects, wherever on the spectrum of amplitude a vibration falls, the vibration also may be characterized by its frequency. In yet further aspects, a low frequency translates in this description as a slow vibration. High frequency is a quick or fast vibration. Thus, as further described herein, a vibration that may be applied by a vibration source or element according to the present invention may be, in various embodiments, strong and slow, weak and quick, strong and quick, weak and slow, and the ranges between.

In further aspects, embodiments of the invention may provide that all vibrations transmitted by the one or more vibration sources of a device have the same frequency but vary in amplitude. In other words, in this aspect, a vibration repeats at the same rate of time, but its strength varies. In other aspects, the opposite may be true in other embodiments of the invention where all vibrations have the same strength (amplitude), but the frequency varies. The level of vibrations in embodiments referenced in this paragraph then, according to some aspects, may be sorted into distinct levels such as low, medium, and high, and/or even to allow for a selection of a level along a continuum between low and high.

In further aspects, in embodiments that allow for variances in both frequency and amplitude of vibration of one or more vibration sources, devices may provide distinct levels of vibration for a user to choose from where each of the levels corresponds to a particular setting of frequency and amplitude. In still further aspects, other embodiments may offer a user a continuum of levels from low to high even as the frequency and/or the amplitude of the vibration changes across the continuum.

According to further embodiments, the invention may provide for more than one level of vibration for the vibration sources. For example, a device according to an embodiment of the invention may include one or more vibration sources with one or more of these sources having more than one level of vibration. In embodiments of the invention that include one or more vibration sources having more than one vibration level, the user of the embodiment may select the level of vibration for the vibration sources in the device. As further described herein, the vibration level selection functionality available to a user may vary from embodiment to embodiment. For example, in further aspects, a user may be able to select a level of vibration, but the same level is selected for all of the vibration sources whether or not the device includes one or more vibration sources. In this aspect, all the vibration sources of that particular embodiment vibrate at the same level. As another example, in other embodiments, a user may be able to select varying or different respective levels of vibration in a device having more than one vibration source.

In still further aspects, a vibrational source can produce a single vibrational cycle, multiple vibrational cycles, or be variable, for example in the vibrations per minute in a particular cycle, or in the number of vibrational cycles. In other words, the vibrational source can be a vibrational motor that operates at, for example, 2,400 vibrations per minute or for another example, at 5,700 vibrations per minute, or in a range from about 2,400 to about 15,000 vibrations per minute, or from about 6,000 to about 15,000 vibrations per minute, or from about 8,000 to about 14,000 vibrations per minute, or from about 9,000 to about 13,000 vibrations per minute, or any vibrations per minute thereinbetween. Alternatively, vibrational source can be a vibrational motor that operates at two or more vibrational cycles, for example, 9,000 vibrations per minute and 13,000 vibrations per minute, and can be switched between vibrational cycles by a switch or other control element. Alternatively, vibrational source can be a vibrational motor that operates at many different vibrational cycles along a continuum by using a potentiostatic switch, for example, vibrational source can be varied continuously or step-wise between 3,000 vibrations per minute and 15,000 vibrations per minute. In an aspect, the vibrational source may provide intermittent vibration cycles, which may be the same or different vibrations per minute. For example, a vibrational source may provide 10,000 vibrations a minute for 4 seconds, stop vibrating for 4 seconds, thus completing one cycle of vibrations, then provide another cycle of vibrations at 10,000 vibrations a minute for 4 seconds, stop vibrating for 4 seconds, and so on. The vibrations per minute may remain the same for each cycle, or may vary randomly or vary in an increasing or decreasing manner. The time of vibration may vary randomly for each cycle, or may vary in an increasing or decreasing manner. The time of no vibration may vary randomly for each cycle, or may vary in an increasing or decreasing manner.

In further aspects, a switch may be a common switch and is used to turn the vibrational source on and off, namely to start and stop the vibration, respectively. The switch may also control power transmission to a control element or other element of the device, such as a sound element or a light. The switch can be secured to the casing at any convenient position where it may readily be actuated, or accessed remotely by wired or wireless components. The switch may be a push button switch located at the anterior side (31) of the device as shown in FIG. 9, or a remote switch connected to an end of a strap as shown in FIG. 12. The switch is electrically connected in a known manner between the power source and the vibrational source to control the application of power to the vibrational source. In an aspect, when the vibrational source is switched on, the vibrating force produced from the vibrational source, such as the various types of motors disclosed above, will be transmitted through the casing to the contacted surface.

In still further aspects, a device of the present invention may have more than one switch, each of which may control the power to an element of the device, or provide on/off control of the element itself, and discussion of one switch is not to be seen as limiting to the invention. A switch can be a common on/off switch, such as a toggle, lever, push-button, capacitance or other switch. For example, a device may be activated by the removal of a barrier so that a circuit is then completed. This completion of the circuit so that the device is activated is a type of switch. The disclosure herein contemplates any type of switch that would be practical with a single vibrational cycle motor. Alternatively, switch can be a common three-way switch. This type of switch would be practical with, a double vibrational cycle motor. Alternatively, a switch can be a common potentiostat. This type of switch would be practical with a vibrational motor that operates at many different vibrational cycles along a continuum. The selection of the type of switch and the control element of a device is within the skill of those knowledgeable in the art. For example, a switch can turn power on or off to a control panel that in turn controls a vibration source, and/or other elements of the device, such as sound or light elements. In some embodiments, the invention may provide application software (an “app”) on a wireless device configured to communicate with each of the vibration sources and/or thermal elements of the device. In further aspects, each of the vibration sources and/or thermal elements may require the appropriate elements to receive, act on the communications from the wireless device, and/or respond to the app with information.

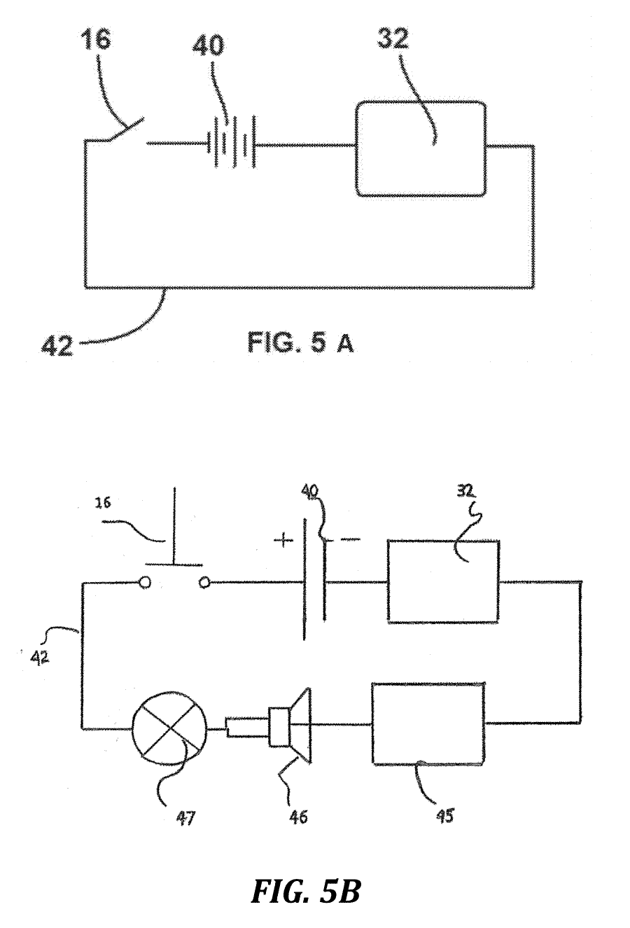

Referring now to FIG. 5A, a representative circuit diagram for the vibrational source is shown. Vibrational source, power source and on/off switch, are electrically connected in series by wiring. Power source is illustrated in FIG. 5 as a battery; however, power source can be any type of power sources such as but not limited to a connection to an alternating current source (a wall plug), a solar or other light cell, a reactor, a mechanical source such as a flywheel or springs, or the like. It is only important that power source be able to provide sufficient power to vibrational source so as to produce sufficient vibration for effecting vibrational vasodilation. FIG. 5B is a circuit diagram of an embodiment of the present invention comprising a vibrational source (32), a push button on/off switch (16), a battery power source (40), a control board (45), and optionally a speaker (46) and a light (47), in electrical connection in series by wiring (42).

As described in further detail herein, in various embodiments, the device may include a controller to receive the communications from the wireless device, to pass them on as instructions to the vibration sources and/or thermal elements, to receive information from the vibration devices, and/or to send the information to the app on the wireless device. The controller may include elements to carry out its function. For example, the controller may include a receiver/transmitter, transceiver, and/or antenna for communicating with the wireless device. The controller may include computing technology such as a microprocessor, etc. to process and execute the instructions, information, and/or signal received from the control unit or wireless unit or the information received from the vibration device. As another example, the controller may be connected respectively by one or more wires (and/or other transmitters or carriers) to the one or more vibration sources and/or thermal elements of the devices to transmit instructions/information to the vibration sources and/or thermal elements and/or to receive information from them.

In operation and use, a device of the present invention is effective in achieving the methods disclosed herein. According to known gate theory, vibration helps to reduce pain as the vibrational or motion nerves surmount the pain nerves. Similarly, it is known that cold helps to reduce pain as the temperature nerves surmount the pain nerves. It also is known that warm thermal contact is effective at vasodilation. It also is believed that vibrational and thermal vasodilation is more effective when applied generally between the pain source or an initiation site and the brain, and more specifically close to the nerve plexi where the various nerve types (pain, temperature and motion) converge in the body, generally at or proximal to a joint.

In further aspects, a thermal element, may be cooled or heated, as described herein. For example, if the thermal element is a metal ingot or low freezing point gel, the thermal element is placed in a refrigerator, freezer, or other cold site. Alternatively, if the thermal element is a high specific-heat capacity material like a grain sewn within an insulated fabric it may be microwaved before use to heat it. When the thermal element is of a satisfactory temperature, the thermal element is placed within or adjacent to the casing. The thermal element may be placed within the thermal element pocket, for example, within an attachment element (50) such as an elastic band attached to the casing so that the thermal element is interposed between the elastic band and the proximal side (30) of the device, or within an attachment element (50) such as a clip located on the proximal side of the device as shown in FIGS. 9A-C and 9E. The device is contacted to the surface, such as the surface of skin of a subject, at the desired location, depending on the method employed for the desired treatment.

In some aspects, such as in the example shown in FIG. 1A where a site of injection is proximal to the subject's wrist, the device is contacted between the injection site and the subject's brain, and more specifically in the illustrative example shown in FIG. 1A, is placed between the initiation site and the subject's elbow and proximal to the nerve plexi proximal to the elbow. In other methods, the device may contact the initiation or injury site directly and not be adjacent to it, as described for certain disclosed methods.

In further aspects, the application area of the device, optionally with the thermal element interposed therebetween, may be applied to the selected area of the subject such that the application area, comprising the thermal area and the vibrational area, contact the subject's skin. The thermal element may be contacted with the surface for a time period, without vibration, for example, to allow the thermal element to act upon the subject for a suitable time period so as to initiate thermal effects, for example, vasodilation or vasoconstriction. Alternatively, concurrently with application of the thermal element to achieve thermal effects, the vibrational source is actuated, for example, by pressing or activating the switch, and starting the vibrational source, and vibrations are transferred through the application area (and through the thermal element if present) to the contacted surface. The vibrational source also is allowed to act upon the subject for a suitable time period so as to initiate the desired effect depending on the method of application. In still further aspects, after thermal and vibrational effects are initiated, a treatment may occur to the subject or the vibrational and thermal effects may be continued until pain or itching sensations are no longer perceived by the subject.

Once the desired treatment is completed, for example, when vessel diameters have been effected, or the sensations are no longer perceived by the subject, the entire device can be removed from contacting the surface, and/or only the thermal element can be removed and the device continues to provide vibration to the surface, or the thermal element may remain in place on the surface and the vibrational source may be turned off. In one illustrative method, the device is left in contact with the subject for a period of between 0 and 60 seconds, or for one or more minutes, to continue to reduce any pain associated with the site.

Referring now to FIGS. 6A and 6B, alternative embodiments include casings having interesting or distracting shapes or ornamental covers (60) over the casing. Distraction may help reduce pain, especially in children. For example, the casing could be a material in the shape of a bumble bee, as illustrated in FIGS. 6A-6B, or dinosaur. When the device is applied to a child, the distracting shape both can lessen the fear a child may have to device medical procedure and help to decrease the subsequent pain or unpleasant sensations of the child.

Referring to FIG. 7, a proximal view of a device 10 of the present invention comprising a thermal element (28) (shown as transparent so as to view the proximal side 30 of the device 10). An attachment element (50) (an elastic band) is shown holding the thermal element (28) to the application area (22) of the proximal side (30) of the casing (12) of the device (10).

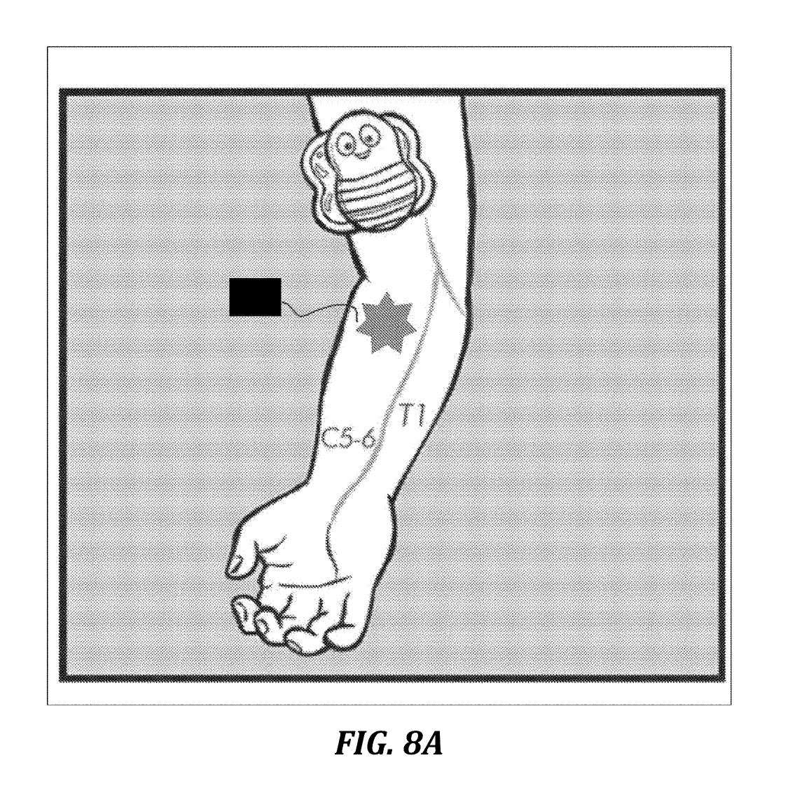

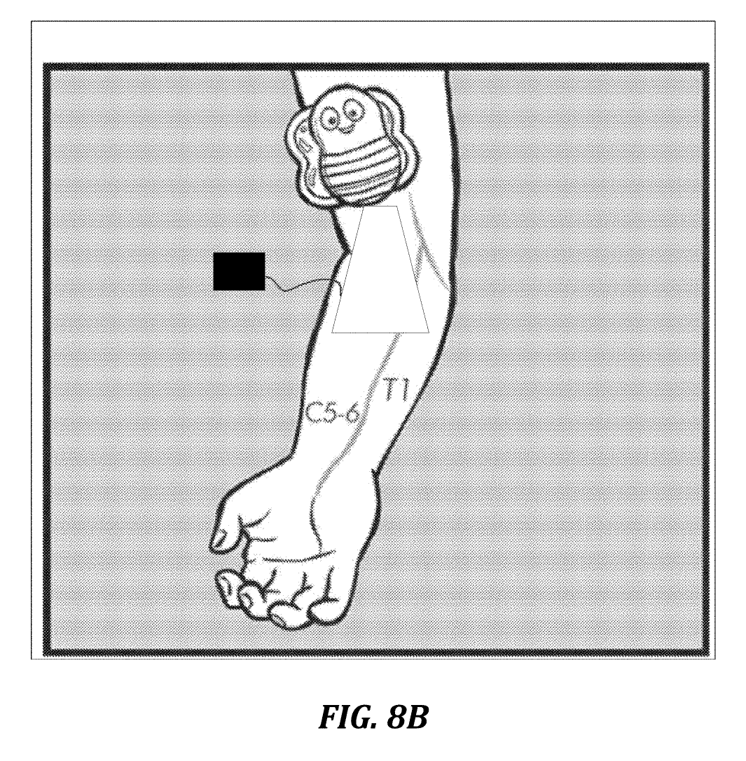

FIG. 8A shows the placement of a device of the present invention (10) on the arm of a human in a location between an initiation site (104a) and the brain/spinal cord of the human. FIG. 8B shows the placement of a device of the present invention (10) on the arm of a human adjacent to an injury site (104b). In various aspects, the device may contact an injury site directly and not be adjacent to it, as described herein for certain disclosed methods.

In various aspects, FIGS. 9A-E show a drawing of an exemplary device of the present invention having a shaped casing. FIG. 9A shows the front or anterior end (31) of the device (10) and its switch (16), FIG. 9B shows the posterior or rear end (33) of the device (10) with its site indicator (52), and FIG. 9C shows the back or proximal side (30) of the device, that is contacts, or is placed proximally or adjacent to, the surface, having a clip (50) for holding a thermal element (not shown) in place. The distal side (30) may be flat or planar in shape, or may be curved, as desired. FIG. 9D shows a front or distal side (37) of the device (10), and FIG. 9E shows a side view (35a or 35b) of the vibratory device (10) where the attachment element (50) a clip, slightly protrudes from the posterior (lower) (33) proximal end and the on/off switch element (16) is shown at the anterior (upper) end (31). The site indicator (52) provides a guide to the user for placing the device. In methods where applicable, the device is placed so that the indicator is directly at the site of injection, pain, treatment or itching sensation. FIG. 9C shows the proximal side (30) of the device 10. For example, the entire proximal side may be the application area (22) and the entire proximal side (30) substantially contacts the surface contacted and the proximal side (30) comprises the application area (22) through which vibration is transferred to the surface. In an aspect, only a portion of the proximal side may be the application area. Also shown is attachment element (52), which is a clip. The indicator (50) is also shown. FIG. 9D shows the distal side 37 of the device which may be curved in a shaped manner to fit comfortably in a hand when gripped. The lateral sides (35a and 35b) are shown as indented in a generally midline position. Other shapes are contemplated by the invention and the indentions may or may not provide a functional or a decorative aspect to the invention. FIG. 9E shows a lateral side (35 a/b) view of the device 10. At the anterior end (31) the switch (16) is visible. The attachment element (clip) (50) is visible on the proximal side (30). The shape of the distal side (37) may be any desired shape, and may be in some embodiments flat or planar, or may be curved, either convex or concave in shape.

In various aspects, the device component characteristics and configuration, such as, for example, size and dimensions, can be configured to adjust for an intended application area, individual user's weight, size, and other factors to achieve optimal vibration and/or thermal transmission and distribution. In further aspects, features of the device and components, such as the vibration source and thermal element, may be configured or utilized to set and/or control the vibrational and/or thermal characteristics. For example, at least one of the following may be configured: the type of material used for the device casing or housing; the vibrational source and/or thermal element dimensions (e.g., height, width, thickness, surface-contacting area, etc.); the thermal characteristics of the thermal elements; the vibration transmission characteristics of the casing material; the thermal conduction characteristics of the casing material; and the extent of exterior surface coverage of surface-contacting portion of the device casing or application area.

In further aspects, while the vibration sources and/or thermal elements described herein can be permanently mounted in or on the device casing or device structure, this is not a requirement. For example, the vibration sources and/or thermal elements can be connected to or disposed within an insert configured to be removably mounted in or on a portion of the device, e.g., to allow interchange and/or replacement of the vibration sources and/or thermal elements. In still further aspects, one or more vibration sources and/or thermal elements can be detachably connected to the casing, e.g., to allow interchange and/or replacement of one or more vibration sources and/or thermal elements (each individually or as a unit with the device casing). Such configurations allow users, medical practitioners, or others to select desired vibrational effects, thermal effects, and pain-reducing properties or levels in the device, e.g., for customization purposes, for personal preferences, to match desired treatment use, a subject's physical characteristics, a subject's symptoms, or to repair or replace defective or damaged device component, etc.

In further aspects, the disclosed devices and systems may further comprise one or more of the following components: a voltage regulator, power switch, power management module, battery management module (e.g., fuel gauge), battery charging module, wireless power coil or receiver, wireless power control module, antenna (e.g., Bluetooth LE antenna), transceiver (e.g., Bluetooth LE transceiver), motor controller, interface module, control module, voltage sensor, current sensor, pulse-width modulation (PWM) module, power input, magnetic switch, motor control module (e.g., vibrational motor control module), and motor drive. In still further aspects, a number of internal components may be mounted within an interior portion of the device and/or casing.

For example, in some embodiments, the device may have an internal component configuration further comprising a controller containing a processing unit, battery back, voltage regulator, power switch, battery management module (e.g., fuel gauge), antenna (e.g., Bluetooth LE antenna), and transceiver (e.g., Bluetooth LE transceiver). In other embodiments, the device may comprise a wireless charging system comprising a battery charging module, wireless power receiver (e.g., wireless power coil), and wireless power control module. In further aspects, a user can simply set the device on a compatible wireless charging mat or cradle to charge the battery in the device.

In further aspects, the disclosed devices may comprise one or more vibration sources and/or thermal elements that can be selectively operable to treat the user. In still further aspects, application software (an app) on a wireless device such as a mobile phone may be used to activate and de-activate and/or otherwise control the vibrations emitted by the one or more vibration sources and/or thermal effects emitted by the one or more thermal elements in the device. In yet further aspects, the disclosed devices may be equipped with wireless technology to communicate with an app on a wireless device. In even further aspects, the app may provide at least ones of: a graphic image of the user's body to indicate where (and/or other characteristics of the vibration and/or thermal effects) the vibration source and/or thermal elements are delivering to the user's body at the time of operation, possible areas of the user's body to indicate where (and/or other characteristics of the vibration and/or thermal effects) the device may be made to deliver vibrations and thermal effects, and telemetry data received from the sensors in the device.

As described herein, the disclosed devices may be made with an inflatable air bladder or chamber in the device. In still further aspects, the casing may comprise an interior air bladder or chamber between the application area and the thermal element and/or vibration source. The walls of the casing may define the air bladder and/or an interior space that, is sized to further contain at least one vibrational element and/or thermal element. In further aspect, the air bladder can be filled or emptied to control the level of thermal effect and/or vibration effect transmitted through the application area to the user. In some aspects, the air bladder or chamber may be filled with a gas to maintain a gap between the application area and thermal element and/or vibration source to prevent or limit the thermal effects or vibration felt by the user through the application area. In other aspects, the air bladder or chamber may be filled with a gas to force the application area and thermal element and/or vibration source against a surface of the user to enhance the thermal effects and/or vibration felt by the user through the application area. In further aspects, the gap distance between the user's body surface and application area can be in the range of from greater than 0.1 mm to about 10 mm. In still further aspects, filling and emptying the air chamber may controlled by a motor control module, motor, actuator, or like device.

In further aspects, the air pocket or chamber can also be configured to provide a predetermined amount of pressure to keep contact between the application area and the user's body surface. In yet further aspects the pressure may be from greater than 0 to about 3.0 kPa. In still further aspects, contact pressure can be sufficient to prevent unwanted transfer of thermal, force, or vibration effects in a given virtual sensation profile, such as may result during operation of the device or from other device components to which it is coupled.

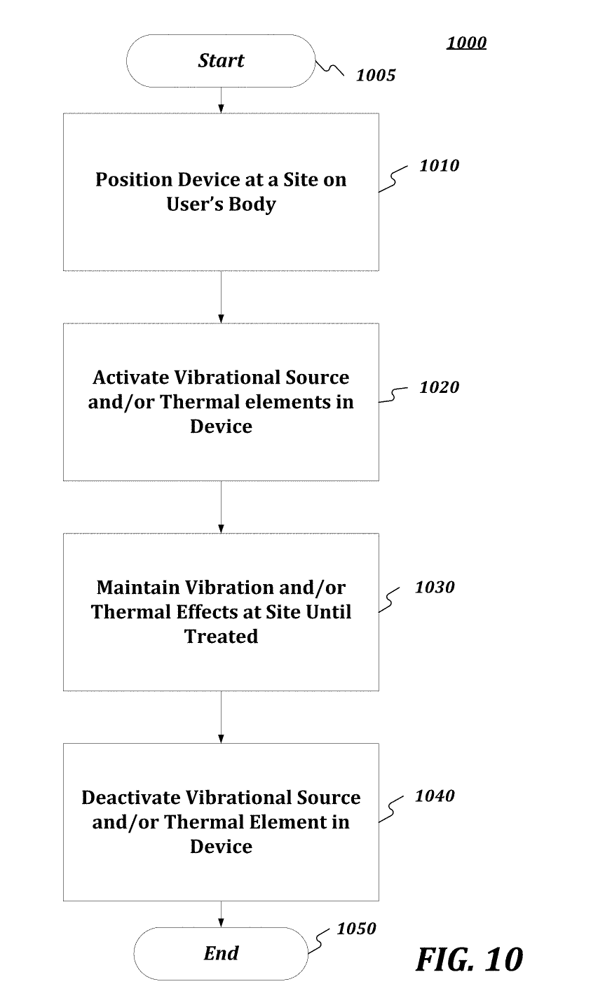

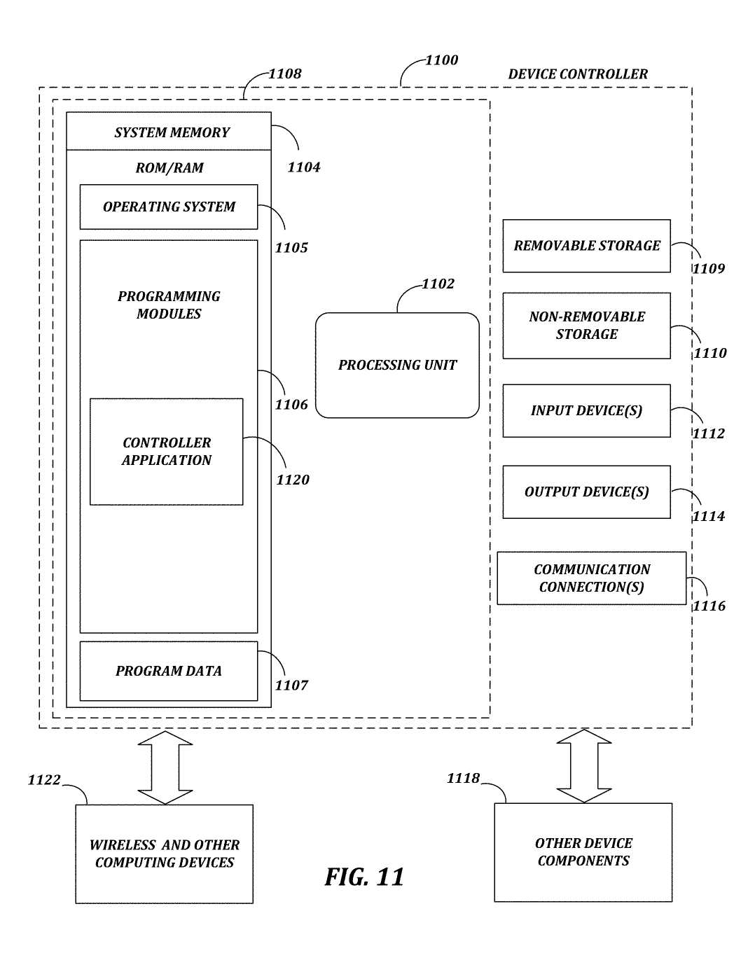

As described herein, also disclosed are various methods of using the disclosed devices to treat a user. For example, in another exemplary aspect, the present disclosure provides a method for healing an injured site and/or reducing pain or discomfort using a disclosed device. FIG. 10 is a flow chart setting forth the general stages involved in a method 1000 consistent with an embodiment of the disclosure for operating the disclosed devices. Method 1000 may be implemented using, at least in part, a controller 1100 (e.g., on-board computing device) as described in more detail below with respect to FIG. 11. Controller 1100 may comprise a controller for operating the device and device components as well as well as performing other operational tasks, including, but not limited to, vibrational control and parameters, thermal control and parameters, and communication. As such, controller 1100 may be in operative configuration and communication with, for example, but not be limited to, vibrational elements, thermal elements, activating switch, communication module, power source, power regulator, various telemetry sensors, transceivers and antennas. As will be detailed with reference to FIG. 11, controller 1100 may comprise a remote communication module to enable remotely operation as described herein. In other embodiments, controller 1100 may be completely self-operating upon configuration.

Furthermore, although stages are disclosed with reference to controller 1100, it should be understood that a plurality of other components may enable the operation of method 1000, including, but not limited to, other computing components, mechanical components, environment properties (e.g., temperature), user conditions, and the like.

Further still, although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein.

Method 1000 may begin at starting block 1005 and proceed to stage 1010, where the device may be placed on a site of a subject. From stage 1010, where the device is positioned on the site of the subject, method 1000 may proceed to stage 1020 where the vibration devices and/or thermal elements may be activated. The activation of device components, though disclosed in a particular order for illustrative purposes, may occur in other arrangements. Upon activation, vibrational sources may begin to vibrate. In some embodiments where present, one or more thermal elements may be activated to provide heat and/or cool to the subject.

In various aspects, an advantage of the invention can be that it allows a user to activate the vibration devices and/or thermal elements in a device remotely from the device. In further aspects, a user does not have to pick up the device to: activate its operation, to shut off operation, and/or in embodiments that allow for adjustment, to adjust the vibration level and/or thermal effect. In still further aspects, a user may activate or de-activate (and/or otherwise control operation of) the vibration source and/or thermal elements by using a control unit, such as a wireless device or mobile device that is in operative communication with the vibration source and/or thermal elements of the device. The wireless device may be a device that may be used for additional purposes other than use with the invention such as a mobile phone, tablet computer, notebook computer, desktop computer, etc. In some embodiments, the invention may provide a specialized wireless device for dedicated use with the invention. In other embodiments, the specialized wireless device may include other uses if its use is not limited to this particular embodiment of the invention.

As provided in more detail herein, the control unit used to control the vibration source and/or thermal elements of the device may include an application or application software (an “app”) specifically created for such usage. Advantageously, the user may download and/or otherwise obtain the app from sources that supply apps such as independent developers and app stores. The app as used with embodiments of the invention communicates wirelessly, such as by using Bluetooth, Wi-Fi, or the like technology.

Controller 1100 (e.g., on-board computing-device) may automatically activate vibrational source and/or thermal elements instantly or after a set amount of time has passed since the launch. In other embodiments, activation may occur upon certain reading from on-board sensors (e.g., including, but not limited to, sensors deployed in the device). For example, activation of the vibrational source and/or thermal elements may be dependent on certain environmental factors and/or user conditions such as, for example, temperature, pulse, blood pressure, acceleration, and the like. Controller 1100 may be configured to trigger activation of various device components upon the satisfaction of certain pre-set conditions. Such conditions may be defined prior to activation.

From stage 1020, where the device components are activated, method 1000 may proceed to stage 1030, where the vibration and/or thermal effects may be maintained at the site of the subject for a time sufficient to treat the subject, such as, for example, to reduce pain or discomfort at the site and/or increase blood flow or healing in the subject. From stage 1030, where the device is used to perform the treatment, method 1000 may proceed to stage 1040, where the device components are turned off. After stage 1040, method 1000 may end at stage 1050.

In various aspects and stages of the disclosed methods, the device may be in operable communication with the user via an antenna or wireless communication component. The user may receive various readings from the various device components. In some embodiments, the user may control the operation of the vibration source and/or thermal elements during use. For example, the user may be able to control the device components, including, but not limited to, vibrational sources or elements, thermal elements, activating switches, communication module, power source, power regulator, various telemetry sensors, transceivers and antennas.

In other embodiments, integrated controller 1100 may be pre-configured with operational control instructions and/or data. In further aspects, embodiments of the device may be used for treating a plurality of sites, such as of injury, pain and/or unpleasant sensations including, but not limited to, burning, itching, and throbbing.

In various aspects, the disclosed devices may comprise, but not be limited to, an integrated controller and/or on-board computing module. The computing module may be in operative configuration and communication with, for example, but not be limited to, vibrational sources or elements, thermal elements, activating switch, communication module, power source, power regulator, various telemetry sensors, transceivers and antennas. Further, the computing module may be in operative communication with another computing device consistent with the description herein, and may comprise, but not be limited to, a wireless device, smart phone, desktop computer, laptop, a tablet, or mobile telecommunications device. Such remote devices may be used to control and/or configure integrated computing module (e.g., activation conditions, vibrational operating parameters and settings, thermal operating parameters and settings and the like).

Moreover, the device may be in operative communication with a centralized server, such as, for example, a cloud computing service. Although operation has been described to be performed, in part, by a controller 1100, it should be understood that, in some embodiments, different operations may be performed by different networked elements in operative communication with controller 1100.

Embodiments of the present disclosure may comprise a system having a memory storage and a processing unit. The processing unit may be coupled to the memory storage, wherein the processing unit is configured to perform the stages of method 1000.

FIG. 11 is a block diagram of a system including controller 1100. Consistent with an embodiment of the disclosure, the aforementioned memory storage and processing unit may be implemented in a computing device, such as controller 1100. Any suitable combination of hardware, software, or firmware may be used to implement the memory storage and processing unit. For example, the memory storage and processing unit may be implemented with controller 1100 or any of vibration sources, thermal elements, and device components 1118, or any other control unit and wireless devices 1122, in combination with controller 1100. Other device components 1118 may comprise, for example, but not be limited to, control mechanisms, vibrational elements, thermal elements, activating switch, communication module, power source, power regulator, various telemetry sensors, transceivers and antennas. The aforementioned system, device, and processors are examples and other systems, devices, and processors may comprise the aforementioned memory storage and processing unit, consistent with embodiments of the disclosure.

With reference to FIG. 11, a system consistent with an embodiment of the disclosure may include a computing device, such as controller 1100. In a basic configuration, controller 1100 may include at least one processing unit 1102 and a system memory 1104. Depending on the configuration and type of computing device, system memory 1104 may comprise, but is not limited to, volatile (e.g. random access memory (RAM)), non-volatile (e.g. read-only memory (ROM)), flash memory, or any combination. System memory 1104 may include operating system 1105, one or more programming modules 1106, and may include a program data 1107. Operating system 1105, for example, may be suitable for controlling controller 1100's operation. In one embodiment, programming modules 1106 may include controller application (“app”) 1120. Furthermore, embodiments of the disclosure may be practiced in conjunction with a graphics library, other operating systems, or any other application program and is not limited to any particular application or system. This basic configuration is illustrated in FIG. 11 by those components within a dashed line 1108.

Advantageously, the app may provide a user with information as well as be the user's interface to operating the embodiment of the invention. The app may include one or more graphic user interfaces (GUIs). Among the GUIs of the app may be a GUI allowing the user to pick which, if there is more than one, vibration source and/or thermal elements to activate, and to select (if available) one or more operating parameters or characteristics (such as amplitude, frequency, and/or temperature) of the vibration source and/or thermal elements of the device. The user may be able to adjust such selections without having to deactivate the embodiment from a GUI of the app. The user may also use the app to turn on and turn off the device components.

The GUI may include additional or other information relating to the vibrations being applied such as the strength (amplitude) or frequency (speed) of the vibrations. The additional or other information may be color coded and/or otherwise presented so as to be readily understood by the user by looking at the GUI of the app. The app may also present the user with information received from the device components, such as environmental and telemetry data.

Controller 1100 may have additional features or functionality. For example, controller 1100 may also include additional data storage devices (removable and/or non-removable) such as, for example, magnetic disks, optical disks, or tape. Such additional storage is illustrated in FIG. 11 by a removable storage 1109 and a non-removable storage 1110. Computer storage media may include volatile and nonvolatile, removable and non-removable media implemented in any method or technology for storage of information, such as computer readable instructions, data structures, program modules, or other data. System memory 1104, removable storage 1109, and non-removable storage 1110 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by controller 1100. Any such computer storage media may be part of device 1100. Controller 1100 may also be operative with input device(s) 1112 such as a keyboard, a mouse, a pen, a sound input device, a touch input device, etc. Input device(s) 1112 may be used to, for example, manually access and program controller 1100. Output device(s) 1114 such as a display, speakers, a printer, etc. may also be included. The aforementioned devices are examples and others may be used.

Controller 1100 may also contain a communication connection 1116 that may allow device 1100 to communicate with other control units and wireless devices 1122 as well as vibration source, thermal elements, and other components 1118 (e.g., transceivers, sensors, thermal elements), such as over an encrypted network in a distributed computing environment. Communication connection 1116 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, Bluetooth, radio frequency (RF), infrared, and other wireless media. The term computer readable media as used herein may include both storage media and communication media.

As stated above, a number of program modules and data files may be stored in system memory 1104, including operating system 1105. While executing on processing unit 1102, programming modules 1106 (e.g., controller application 1120) may perform processes including, for example, one or more of stages or portions of stages of method 1000 as described above. App 1120 may be configured to operate device components 1118 and receive instructions from, for example, communications connections module 1116. The aforementioned process is an example, and processing unit 1102 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Thus, in exemplary embodiments, the invention is a device for providing vibration and/or thermal treatment to a surface, comprising a casing comprising an application area, wherein at least a portion of the application area is shaped to substantially contact a surface, such as a subject's skin, a vibrational source contained within the casing, with said vibrational source capable of producing vibration that is transferred through the casing to at least the surface, and optionally comprising a thermal element capable of transmitting heat or cold. The application area is constructed to allow the transmission of vibration from the vibrational source to the surface, such as a subject's skin, and by the interpositioning of a thermal element or source between at least two of: a vibration area, an application area and the surface, providing thermal effects to the surface. The vibration or combination of the vibration and transmission of cold or heat from the thermal element produces vibrational and thermal effects on the subject.

In further aspects, the invention further comprises the use of a removable thermal element. For example, in some aspects, the casing may comprise a flat hook on which a thermal element or pack could be attached while still transmitting vibrational energy if the pack were soft. In other aspects, the casing may comprise a band attached to an outer surface of the casing; where a cavity is formed between the band and casing which defines an inner space of a thermal element pocket in which a thermal element or pack could be placed in while still transmitting vibrational energy if the pack were soft.

In yet further aspects, the invention further comprises a vibrational source or unit with a power source capable of being attached via an adhesive or adhesive dressing (e.g. tegaderm) or attached to the skin in an array of vibrational units.

In still further aspects, the present invention may comprise a kit comprising a vibratory device of the present invention, a thermal element and instructions for use of the device, for example, in connection with treatment of a subject.

As used herein, subject means a human or animal, and includes any living animal on the planet Earth.

As used herein the singular forms “a”, “and”, and “the” include plural referents unless the context clearly dictates otherwise. All technical and scientific terms used herein have the same meaning as commonly understood to one of ordinary skill in the art to which this invention belongs unless clearly indicated otherwise.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

The present invention includes at least the following aspects: Aspect 1: A vibratory device for treating a site of a subject, the device comprising: a) a casing comprising a thermal element pocket and an application area; b) at least one vibrational source contained within the casing, the at least one vibrational source being capable of producing vibration; c) a switch in operative communication with the at least one vibrational source for controlling operation of the at least one vibrational source, and d) a thermal element removably contained within the thermal element pocket, the thermal element being capable of producing thermal effects selected from at least one of heat or cold.

Aspect 2: The device of any preceding aspect, wherein the thermal element pocket comprises an application area configured to allow transfer of thermal effects from the thermal element to a surface of the site of the subject.

Aspect 3: The device of any preceding aspect, wherein at least a portion of the application area corresponds to at least a portion of an outer surface of the thermal element pocket.

Aspect 4: The device of any preceding aspect, wherein substantially all of a surface of the thermal element is in thermal communication with the application area.

Aspect 5: The device of any preceding aspect, further comprising a band attached to an outer surface of the casing; and wherein a cavity formed between the band and casing defines the thermal element pocket for containing the thermal element.

Aspect 6: The device of any preceding aspect, further comprising a band attached to an outer surface of the casing; and wherein a cavity formed between the band and casing defines a space within the thermal element pocket for containing the thermal element.

Aspect 7: The device of any preceding aspect, wherein at least a portion of the thermal element pocket is comprised of a thermally conductive and rigid material capable of transmitting vibration.

Aspect 8: The device of any preceding aspect, wherein the at least one vibration source is disposed on an interior surface of the application area of the thermal element pocket.

Aspect 9: The device of any preceding aspect, wherein at least a portion of the band of the thermal element pocket is comprised of a thermally conductive and rigid material.

Aspect 10: The device of any preceding aspect, wherein at least a portion of the band of the thermal element pocket is comprised of a thermally conductive and rigid transmission plate.

Aspect 11: The device of any preceding aspect, wherein at least a portion of an outer wall of the thermal element pocket comprises at least a portion of the application area.

Aspect 12: The device of any preceding aspect, wherein the thermal element is secured by at least one of friction, adhesive, hook, clip, and knob.

Aspect 13: The device of any preceding aspect, wherein the thermal element is secured within the thermal element pocket by at least one of: friction, adhesive, hook, clip, and knob.

Aspect 14: A vibratory device for treating a site of a subject, the device comprising: a) a casing comprising an application area; b) at least one vibrational source contained within the casing, wherein the at least one vibrational source is capable of producing vibration; c) a switch in operative communication with the at least one vibrational source for controlling operation of the at least one vibrational source; and d) a thermal element secured to the casing, the thermal element being capable of producing thermal effects selected from at least one of hot or cold.

Aspect 15: The device of the preceding aspect, wherein the thermal element is secured to the casing by at least one attachment element.

Aspect 16: The device of the preceding aspect, wherein the at least one attachment element comprises at least one of an adhesive, solder, weld, hook, clip, clasp, and knob.

Aspect 17: The device of any preceding aspect, wherein the device is configured to transfer vibration from the vibration source to the site's surface through the application area.

Aspect 18: The device of any preceding aspect, wherein the vibration is transferred to the site from the application area through the thermal element.

Aspect 19: The device of any preceding aspect, wherein the thermal effects are transferred to the site from the application area through the vibration source.

Aspect 20: The device of any preceding aspect, wherein the vibrational source is in mechanical communication with the thermal element such that substantially all the vibration from the vibration source is transferred through the thermal element.

Aspect 21: The device of any preceding aspect, wherein the vibrational source is in mechanical communication with the thermal element such that all the vibration from the vibration source is transferred through the thermal element.

Aspect 22: The device of any preceding aspect, wherein at least a portion of the application area corresponds to at least a portion of an outer surface of the thermal element.

Aspect 23: The device of any preceding aspect, wherein the thermal element is in thermal communication with at least a portion of the application area.

Aspect 24: The device of any preceding aspect, wherein the vibrational source and thermal source are operatively positioned relative to one another such that the vibrational source and thermal element cooperate to provide vibration and thermal effects to the site of the subject.

Aspect 25: The device of any preceding aspect, wherein the device is configured to provide thermal effects simultaneously with vibration; and wherein the application area is configured to allow transfer of vibration from the vibration source to a surface of the site of subject.

Aspect 26: The device of any preceding aspect, wherein the at least one vibration source is configured to allow transfer through of thermal effects from the thermal element to a surface of the site of subject.

Aspect 27: The device of any preceding aspect, wherein the device is configured to provide a thermal effect simultaneously with vibration; and wherein the application area is configured to allow transfer of vibration and thermal effects to a surface of the site of subject.

Aspect 28: The device of any preceding aspect, wherein the device is configured to deliver at least one thermal effect simultaneously with the vibration that is effective to treat a site in a subject.

Aspect 29: The device of any preceding aspect, wherein the combination of vibration and thermal effects transmitted produces a vibrational analgesia and thermal analgesia treatment effective to reduce pain associated with the site of the subject.

Aspect 30: The device of any preceding aspect, wherein the combination of vibration and thermal effects transmitted produces a vibrational treatment and thermal treatment effective to improve a wound associated with the site of the subject.

Aspect 31: The device of any preceding aspect, wherein the combination of vibration and thermal effects transmitted produces a vibrational treatment and thermal treatment effective to enhance healing associated with the site of the subject.

Aspect 32: The device of any preceding aspect, wherein at least a portion of the casing is comprised of a thermally conductive and rigid material.

Aspect 33: The device of any preceding aspect, wherein the thermally conductive and rigid material comprises at least one of metal and thermally-conductive thermoplastic compositions.

Aspect 34: The device of any preceding aspect, wherein the thermal element is configured to modulate a temperature of a contacted area associated with the site of the subject.

Aspect 35: The device of any preceding aspect, wherein the thermal element is constructed to allow vibration to transfer through the thermal element.

Aspect 36: The device of any preceding aspect, wherein the thermal element comprises at least one of metal ingots, ice packs, low freezing point liquid packs, low freezing point gel packs, ceramics, heat sinks, Peltier coolers, thermoelectric heat pumps, and thermoelectric coolers.

Aspect 37: The device of any preceding aspect, wherein the thermal element comprises at least one of heat packs, chemical reactive thermal packs, thermal gel packs, thermal clay packs, calcium chloride-based heat packs, supersaturated sodium acetate-based heat packs, Peltier heaters, electric heating elements, metal heating elements, ceramic heating elements, polymer positive temperature coefficient (PTC) heating elements, and composite heating elements.

Aspect 38: The device of any preceding aspect, wherein the vibration is initiated by activating the vibrational source using the switch.

Aspect 39: The device of any preceding aspect, wherein the thermal element comprises at least one of Peltier coolers, thermoelectric heat pumps, thermoelectric coolers, Peltier heaters, and electric heating elements; and wherein the device is further configured to initiate at least one thermal effect by activating the thermal element using a switch.

Aspect 40: The device of any preceding aspect, wherein the switch is configured to allow selective activation of the vibrational source while the device is secured to a subject.

Aspect 41: The device of any preceding aspect, wherein the switch is configured to allow selective activation of the thermal while the device is secured to a subject.

Aspect 42: The device of any preceding aspect, wherein the switch comprises a remote switch configured to remotely activate at least one of the at least one vibrational source and thermal element.

Aspect 43: The device of any preceding aspect, wherein the switch comprises a wireless device configured to wireless activate at least one of the vibrational source and thermal element.

Aspect 44: The device of any preceding aspect, wherein the wireless switch comprises a wireless device configured to transmit instructions regarding operation of the vibratory device.

Aspect 45: The device of any preceding aspect, wherein the wireless device is operational to at least one of: present information regarding the operation of the vibratory device to a user via one or more graphic user interfaces on the wireless device, receive instructions regarding operation of the vibratory device from a user, and transmit the instructions to the vibratory device.

Aspect 46: The device of any preceding aspect, wherein the wireless switch comprises a wireless device having application software configured to transmit instructions regarding operation of the vibratory device.

Aspect 47: The device of any preceding aspect, wherein the application software is operational to at least one of: present information regarding operation of the vibratory device to a user via one or more graphic user interfaces on the wireless device, receive instructions regarding operation of the vibratory device from a user, and transmit the instructions to the vibratory device.

Aspect 48: The device of any preceding aspect, wherein operation of the vibratory device comprises at least one of selective activation of the vibrational source, selective activation of the thermal element, selection of a parameter associated with the vibration, selection of a parameter associated with at least one thermal effect; and selection of an activation duration.

Aspect 49: The device of any preceding aspect, wherein a vibration parameter comprises at least one of a continuous vibration cycle, an intermittent vibration cycle, a vibration per minute associated with the vibration, a frequency associated with the vibration, or an amplitude associated with the vibration.

Aspect 50: The device of any preceding aspect, wherein a thermal effect parameter comprises at least one of a thermal effect cycle and a temperature associated with at least one thermal effect.

Aspect 51: The device of any preceding aspect, wherein the vibration source is configured to deliver vibration in a single vibration cycle or multiple vibration cycles.

Aspect 52: The device of any preceding aspect, wherein a vibration per minute of the vibration is constant or variable.

Aspect 53: The device of any preceding aspect, wherein the vibration per minute of the vibration is 2,400 to 15,000 vibrations per minute.

Aspect 54: The device of any preceding aspect, wherein the vibration is variable and cycles between 6,000 to 15,000 vibrations per minute.

Aspect 55: The device of any preceding aspect, wherein the vibration frequency is from about 2,400 RPM to about 6,000 RPM.

Aspect 56: The device of any preceding aspect, wherein the vibration amplitude is from about 0.5 G to about 8 G.

Aspect 57: The device of any preceding aspect, further comprising a strap for securing the casing to the site of the subject.

Aspect 58: The device of any preceding aspect, further comprising a strap for securing the casing. such that the application area is spaced a predetermined distance from the site of the subject.

Aspect 59: The device of any preceding aspect, wherein the casing is dimensioned and shaped to be secured along a curvature of at least one of a subject's back, shoulder, and/or neck.

Aspect 60: The device of any preceding aspect, wherein the casing is dimensioned and shaped to be secured along a curvature of a subject's back.

Aspect 61: The device of any preceding aspect, wherein the casing is dimensioned and shaped to be secured along a curvature of a subject's shoulder.

Aspect 62: The device of any preceding aspect, wherein the casing is dimensioned and shaped to be secured along a curvature of a subject's neck.

Aspect 63: The device of any preceding aspect, wherein the casing is dimensioned to have a length of from about 3 inches to about 36 inches.

Aspect 64: The device of any preceding aspect, wherein the casing is dimensioned to have a width of from about 3 inches to about 12 inches.

Aspect 65: The device of any preceding aspect, wherein the casing is dimensioned to have a length of from about 3 inches to about 36 inches, and a width of from about 3 inches to about 12 inches; and wherein the casing is shaped to have a convex shape adapted to be secured along a curvature of a subject's back.

Aspect 66: The device of any preceding aspect, wherein the application area is constructed to transmit the thermal effects from the thermal element to the subject's skin and to transmit vibration from the vibrational source to the subject's skin,

Aspect 67: The device of any preceding aspect wherein the site comprises at least one of a site of pain and/or a site of irritation.

Aspect 68: The device of any preceding aspect wherein the site comprises at least one of a site of injury and/or a site of a wound.

Aspect 69: The device of any preceding aspect, wherein the thermal element is capable of modulating the subject's skin to a temperature of between about 15° F. (−9.4° C.) and about 120° F. (48.9° C.).

Aspect 70: The device of any preceding aspect, wherein the thermal element is capable of cooling the subject's skin to a temperature of below about 45° F. (7.2° C.).

Aspect 71: The device of any preceding aspect, wherein the thermal element is capable of cooling the subject's skin to a temperature of between about 15° F. (−9.4° C.) and about 28° F. (−2.20 C).

Aspect 72: The device of any preceding aspect, wherein the thermal element is capable of heating the subject's skin to a temperature of at least about 120° F. (48.9° C.).

Aspect 73: The device of any preceding aspect, wherein the thermal element is capable of heating the subject's skin to a temperature of between about 95° F. (35° C.) and about 120° F. (48.9° C.).

Aspect 74: The device of any preceding aspect, further comprising a plurality of vibration sources.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

The above detailed description of the preferred embodiments, and the examples, are for illustrative purposes only and are not intended to limit the scope and spirit of the invention, and its equivalents, as defined by the claims. One skilled in the art will recognize that many variations can be made to the invention disclosed in this specification without departing from the scope and spirit of the invention.

### REFERENCES

Baxter A L, Leong T, Mathew B. External Thermomechanical Stimulation versus Vapocoolant for Adult Venipuncture Pain: Pilot Data on a Novel Device. Clin J Pain 2009; 25(8):705-710.

Baxter A L, Cohen L L, Lawson M L, Von Baeyer C L. A Randomized Clinical Trial of a Novel Vibrating Tourniquet to Decrease Pediatric Venipuncture Pain. Pediatr Emerg Care 2011; 27(12): 1151-6.

Bovenzi M, Lindsell C J, Griffin M J. Duration of acute exposures to vibration and finger circulation. Scand J Work Environ Health 1998; 24(2): 130-7.

Figueroa A, Gil R, Sanchez-Gonzalez M A. Whole-body vibration attenuates the increase in leg arterial stiffness and aortic systolic blood pressure during post-exercise muscle ischemia. Eur J Appl Physiol 2011; 111(7):1261-8.

Figueroa A, Gil R, Wong A, Hooshmand S, Park S Y, Vicil F, et al. Whole-body vibration training reduces arterial stiffness, blood pressure and sympathovagal balance in young overweight-obese women. Hypertens Res 2012.

Freeman A W, Johnson K O. A model accounting for effects of vibratory amplitude on responses of cutaneous mechanoreceptors in macaque monkey. J Physiol 1982; 323:43-64.

Hess H A. A biomedical device to improve pediatric vascular access success. Ped Nurs 2010; 36(5): 259-263.

Inal S, Kelleci M. Buzzy relieves pediatric venipuncture pain during blood specimen collection. MCN Am J Matem Child Nurs 2012; in press.

Skoglund C R. Vasodilatation in human skin induced by low-amplitude high-frequency vibration. Clin Physiol 1989; 9(4):361-72.

Spandorfer P R, Alessandrini E A, Joffe M D, Localio R, Shaw K N. Oral versus intravenous rehydration of moderately dehydrated children: a randomized, controlled trial. Pediatrics 2005; 115(2):295-301.

Taddio A, Soin H K, Schuh S. Koren G, Scolnik D. Liposomal lidocaine to improve procedural success rates and reduce procedural pain among children: a randomized controlled trial. Cmaj 2005:172(13): 1691-5.

Thompson A J, Griffin M J. Effect of the magnitude and frequency of hand-transmitted vibration on finger blood flow during and after exposure to vibration. Int Arch Occup Environ Health 2009; 82(9):1151-62.

Dehghan M and Farahbod F. The Efficacy of Thermotherapy and Cryotherapy on Pain Relief in Patients with Acute Low Back Pain, A Clinical Trial Study. J Clin Diagn Res. 2014 September; 8(9): LC01-LC04.

Lurie R C, Cimino S, Gregory D E, Brown S H M. The effect of short duration low back vibration on pain developed during prolonged standing. Applied Ergonomics 67 (2018) 246-251.

What is claimed:

1. A vibratory device for treating a site of a subject, the device comprising:
a) a casing comprising a thermal element pocket and an application area;
b) at least one vibrational source contained within the casing, the at least one vibrational source being capable of producing vibration;
c) a switch in operative communication with the at least one vibrational source for controlling operation of the at least one vibrational source, and
d) a thermal element removably contained within the thermal element pocket, the thermal element being capable of producing thermal effects selected from at least one of heat or cold.

2. The device of claim 1, wherein the thermal element pocket comprises an application area configured to allow transfer of thermal effects from the thermal element to a surface of the site of the subject and vibration from the at least one vibrational source to the surface of the site of the subject.

3. The device of claim 2, further comprising a band attached to an outer surface of the casing; and wherein a cavity formed between the band and casing defines the thermal element pocket for containing the thermal element.

4. The device of claim 2, wherein at least a portion of the thermal element pocket is comprised of a thermally conductive and rigid material capable of transmitting vibration and thermal effects.

5. The device of claim 4, wherein the thermal element is secured by at least one of friction, adhesive, hook, clip, and knob.

6. The device of claim 5, wherein the device is configured to provide thermal effects simultaneously with vibration; and wherein the application area of the thermal pocket is configured to allow simultaneous transfer of thermal effects from the thermal element and vibration from the vibration source to a surface of the site of subject.

7. The device of claim 6, wherein the combination of vibration and thermal effects transmitted produces a vibrational analgesia and thermal analgesia treatment effective to at least one of reduce pain associated with the site of the subject, improve a wound associated with the site of the subject, and enhance healing associated with the site of the subject.

8. A vibratory device for treating a site of a subject, the device comprising:
a) a casing comprising an application area;
b) at least one vibrational source contained within the casing, wherein the at least one vibrational source is capable of producing vibration;
c) a switch in operative communication with the at least one vibrational source for controlling operation of the at least one vibrational source; and
d) a thermal element secured to the casing, the thermal element being capable of producing thermal effects selected from at least one of hot or cold.

9. The device of claim 8, wherein the thermal element is secured to the casing by at least one attachment element.

10. The device of claim 9, wherein the device is configured to transfer vibration from the vibration source to the site's surface through the application area.

11. The device of claim 10, wherein the vibration is transferred to the site from the application area through the thermal element.

12. The device of claim 10, wherein the at least one vibrational source is in mechanical communication with the thermal element such that all the vibration from the vibration source is transferred through the thermal element.

13. The device of claim 12, wherein the device is configured to provide thermal effects simultaneously with vibration; and wherein the application area is configured to allow transfer of vibration from the vibration source to a surface of the site of subject.

14. The device of claim 13, wherein the combination of vibration and thermal effects transmitted produces a vibrational analgesia and thermal analgesia treatment effective to at least one of reduce pain associated with the site of the subject, improve a wound associated with the site of the subject, and enhance healing associated with the site of the subject.

15. The device of claim 13, wherein at least a portion of the casing is comprised of a thermally conductive and rigid material.

16. The device of claim 14, wherein the thermal element comprises at least one of Peltier coolers, thermoelectric heat pumps, thermoelectric coolers, Peltier heaters, and electric heating elements; and wherein the device is further configured to initiate at least one thermal effect by activating the thermal element using the switch.

17. The device of claim 14, wherein the switch comprises a wireless device having application software configured to transmit instructions regarding operation of the device; and the application software is operational to at least one of: present information regarding operation of the vibratory device to a user via one or more graphic user interfaces on the wireless device, receive instructions regarding operation of the vibratory device from a user, and transmit the instructions to the vibratory device.

18. The device of claim 17, wherein operation of the vibratory device comprises selection of a parameter associated with vibration; and wherein a vibration parameter comprises at least one of a continuous vibration cycle, an intermittent vibration cycle, a vibration per minute associated with the vibration, a frequency associated with the vibration, or an amplitude associated with the vibration.

19. The device of claim 13, wherein the vibration has a vibration amplitude from about 0.5 G to about 8 G.

20. The device of claim 13, wherein the casing is dimensioned and shaped to be secured along a curvature of at least one of a subject's back, shoulder, or neck.
